(19) United States
(12) Patent Application Publication
(10) Pub. No.: US 2004/0138535 A1
(43) Pub. Date: Jul. 15, 2004

(54) Recreational facility with security and medical screening

(76) Inventor: John W.L. Ogilvie, Salt Lake City, UT (US)

Correspondence Address: JOHN W. L. OGILVIE, 1320 EAST LAIRD AVENUE, SALT LAKE CITY, UT, 84105, US

(21) Appl. No.: 10/384,904

(22) Filed: Mar. 7, 2003

Related U.S. Application Data
(60) Provisional application No. 60/440,413, filed on Jan. 13, 2003.
(60) Provisional application No. 60/443,630, filed on Jan. 29, 2003.
(60) Provisional application No. 60/448,514, filed on Feb. 20, 2003.

(51) Int. Cl. A61B 5/00

(52) U.S. Cl. 600/300

(57) ABSTRACT

The present invention provides tools and techniques for recreational facilities (100) in which medical screening (710) results are made known to facility patrons, thereby permitting better-informed recreational interactions. Facilities (100) may include medical screening regions (112), recreational regions (114, 116, 118), exit processing regions (130) and other regions. Methods for use in recreational facilities may include medical screening (710, 810), permissioning (724, 824), entry (726, 826) to recreational regions, sharing (730, 830) of medical screening results through badges (500), displays (238), or other means, recreational activities (742, 744, 842, 844), and other steps.

### RELATED APPLICATIONS

This application claims priority to, and the benefit of, and incorporates by reference, several prior patent applications, namely, U.S. patent application serial No. 60/440,413 filed 13 Jan. 2003, U.S. patent application serial No. 60/443,630 filed 29 Jan. 2003, and U.S. patent application serial No. 60/448,514 filed 20 Feb. 2003.

### FIELD OF THE INVENTION

The present invention relates generally to three categories of subject matter, the first subject being amusement, entertainment, and recreation in a building or other facility (see, e.g., the definition of U.S. patent class 472 subclass 136), the second subject being medical testing (see, e.g., definitions of U.S. class 435 subclasses 7.36 and 806), and the third subject being security-related technologies (see, e.g., definitions of U.S. classes 40, 70, 116, 705). Other subjects are also involved. The invention relates in particular to tools and techniques which facilitate adult recreation in a secured facility by making medical test results known to patrons of the facility.

### BACKGROUND OF THE INVENTION

Sex is not inherently a bad thing; in many cases, it is good both for individuals and for society. But it has long been recognized that sexual activity can cause great harm, by spreading sexually transmitted diseases (STDs) and by conceiving unwanted children, for example. Various approaches have been proposed to reduce the occurrence of either or both of these harms, such as: sexual abstinence, restricting sexual activity to sex between people who are married to each other, restricting sexual activity to monogamous relations (whether in marriage or otherwise), sterilization, forms of contraception which are less permanent than sterilization, and/or the use of prophylactics such as condoms and dental dams. As used herein, to “reduce” something means either to merely decrease it or to completely eliminate it.

In addition to the approaches just listed, it will be recognized that certain types of sexual activity can have the side-effect of reducing STDs and/or unwanted pregnancies, even if the participants do not choose the activities for that reason. Examples comprise activities that provide sexual satisfaction without necessarily involving a transfer of bodily fluid between participants.

It must also be acknowledged that the sex drive is a powerful force in human behavior. Even when people understand that sexual activity poses some risk of disease transmission, unwanted pregnancy, or other harmful consequences, they often choose to engage in such activity despite the risks. The risks are often perceived as vague or distant, while the desires that influence choices about personal behavior are quite specific, nearby, and strong.

In addition, choices about sex are often very personal and may be quite emotional. They may reflect strongly held religious or moral convictions. They are also made in the context of social policies and criminal laws that are meant to guide choices about sex in various localities and countries. In addition, different people face different circumstances and have different goals.

The variety of sex-related choices people make is illustrated by the enormous range and number of materials available on the Internet, which includes for example: sites advocating sexual abstinence; governmental and private sector sites aimed at educating people about sexually transmitted diseases; sites that provide information about adverse consequences of risky sex, such as unwanted pregnancies, sexually transmitted diseases, and life upheaval; sites that describe and/or sell sexually explicit books, movies, adult toys, and other accoutrements of some sexual behaviors; sites devoted to and/or discussing HIV+ people; sites devoted to and/or discussing intersexual people; sites devoted to and/or discussing gay and/or lesbian people; sites that advertise private clubs or resorts for adults; sites that advertise escort services; sites that describe or advertise sexual services; and sites that link to other sites having text, pictures, videos, chat rooms, adult toys, and/or other materials that are intended for a wide range of sexual preferences (e.g., www.persiankitty.com and sites identified therein). There is also some overlap between discussions of sexually transmitted diseases and discussions of other contagious diseases, since both concern personal and public health.

Because of all this, approaches that work well in one situation or one jurisdiction may not serve to deter or prevent potentially harmful activity in other circumstances.

Accordingly, it would be worthwhile to consider new approaches that might help reduce the spread of disease, the incidence of unwanted pregnancies, or both. The present invention is proposed and described with that goal in mind.

### BRIEF SUMMARY OF THE INVENTION

The invention provides tools and techniques which can help make recreation safer by encouraging disclosure of medical information so that people can make informed choices. In some situations, the invention may help dissuade people from participating in risky activities by helping them better understand the actual risks involved. In some situations, the invention may help people obtain recreational satisfaction by helping them identify activities that pose little or no risk to them or others, which might not have been identified without the invention. Different embodiments and uses of the invention may serve different goals for different people, so this summary is provided to help illustrate the invention, not as a replacement for the claims that define the invention. The present invention is defined by the claims, and to the extent this summary conflicts with the claims, the claims should ultimately prevail.

In some embodiments, the invention provides a recreational facility having at least three regions, namely, a secured recreational region, a medical screening region, and an authentication region. Methods, facilities, and devices according to the invention may facilitate consensual sexual activity between adults in the recreational region and/or other recreational activities. Within the medical screening region, a medical screening means is provided for screening patrons of the recreational facility to determine their medical status with respect to at least one of two sex-related conditions, namely, sexually transmitted disease status and fertility status. In addition or alternately, screening is done in some facilities to check for contagious diseases that are not typically sexually transmitted. The medical screening region may include a physical examination room, a medical counseling office, and/or a lab for processing medical tests. Within the authentication region, an authentication means is provided for authenticating employees of the recreational facility, other representatives of the facility, and other authorized persons, and thus for detecting unauthorized persons through a lack of authentication.

Physical security means separate the secured recreational region from the medical screening region and define a secured path containing the medical screening region and the secured recreational region. The secured path is configured to allow patrons of the recreational facility to enter the secured recreational region after passing through the medical screening region, and to inhibit patrons from entering the secured recreational region without first passing through the medical screening region. A security perimeter encompasses at least the secured recreational region, the medical screening region, and the authentication region. The security perimeter is defined by physical security barriers that operate to direct patrons such that a patron who leaves the regions encompassed by the security perimeter can return to the secured recreational region only after again passing through the medical screening region, and that operate to inhibit unauthorized persons from entering the secured recreational region.

In some embodiments, the recreational facility also has an exit processing region which has an exit at the security perimeter and an entrance adjoining the secured recreational region. Physical security means in the facility operate to direct patrons such that a patron who enters the exit processing region from the secured recreational region can return to the secured recreational region only after again passing through the medical screening region.

In some embodiments, the recreational facility also includes one or more of the following: a lodging region which includes private rooms for patrons to use while they stay overnight in the secured recreational region; an eating region which includes furniture for patrons to use while they eat food in the secured recreational region; a dancing region which includes a dance floor for patrons to use while they dance in the secured recreational region; a physically demarcated preference zone in the secured recreational region for patrons to use while they participate in recreational activities; a physically demarcated and secured requirements zone for patrons to use while they participate in recreational activities in the secured recreational region; a locker room outside the secured recreational region with lockable storage to hold personal belongings of patrons before they enter the secured recreational region; a display that is readily visible inside the secured recreational region, that is not readily visible outside the security perimeter, and is capable of displaying to patrons in the recreational region medical status information of other patrons, obtained within the medical screening region.

In some embodiments, the facility's construction and physical security serve to define a secured path in which a reception region is followed by the medical screening region which is followed by the secured recreational region. Thus, patrons enter the secured recreational region only after passing through the reception region and then passing through the medical screening region. In some embodiments, the secured path includes an exit processing region following the secured recreational region. In these embodiments, patrons normally exit the facility only through the exit processing region, and having entered the exit processing region are barred from returning to the secured recreational region except by passing again through the reception region and through the medical screening region where they are again medically screened.

In some embodiments, the invention provides a secured medical screening method for improving recreational activities. A medical screening region and a recreational region are provided within a security perimeter. Then people in the medical screening region are medically screened to determine their medical status with respect to at least one of three conditions, namely, sexually transmitted disease status, other contagious disease status, and fertility status. Screening may be done by medical testing in the recreational facility, or it may be done by verifying tests, surgeries, and other medical events done outside the facility, or it may be done using a mixture of these two approaches. Verification of medical status may be done, for instance, by telephone or other authorized and reliable communication with a doctor's office, clinic, or hospital, and/or by checking official databases if such exist. Counseling services may be provided to help people understand and adapt to their screening results.

Some methods medically screen people with respect to at least their sexually transmitted disease (STD) status, and then allow medically screened people to enter the recreational region only if they satisfy an STD compatibility criterion. Facility employees are not necessarily screened or required to have STD compatibility, although they may be screened for STD and/or fertility status, since employees may be prohibited from sexual and/or other activities that are likely to transfer bodily fluids between them and patrons. One STD compatibility criterion requires that all patrons in the recreational region have a single specified STD status, and two patrons have the same STD status when exactly one of the following is true: each patron has no STDs as determined by the medical screening; each patron has the same STD as determined by the medical screening; each patron has the same multiple STDs as determined by the medical screening. A facility may have multiple recreational regions, some of which are subject to different STD compatibility criteria than others. Transfer of bodily fluids between any two patrons who have different STDs may be prohibited in the recreational facility.

Some methods medically screen people with respect to at least their fertility status, and then allow medically screened people to enter the recreational region only if they satisfy a fertility compatibility criterion. Facility employees are not necessarily screened or required to have fertility compatibility, since they may be prohibited from activities that are likely to conceive offspring. One fertility compatibility criterion requires that all patrons in the recreational region as a group meet at least one of the following criteria: each patron is the same gender as the other patrons in the recreational region; each patron in the recreational region is infertile; no patron in the recreational region is fertile as a male; no patron in the recreational region is fertile as a female. A facility may have multiple recreational regions, some of which are subject to different fertility compatibility criteria than others. Transfer of bodily fluids between any two fertile patrons may be prohibited in the recreational facility.

People whose medical status has been determined by the medical screening can be admitted to the recreational region. In some embodiments, people are admitted only if a badge or other device will readily display their screening results to other people in the recreational region. People who have not been medically screened can be barred from the recreational region. Professional sex workers such as prostitutes may be barred from the recreational region, regardless of whether they have been medically screened.

Recreational activities by medically screened people can be facilitated within the recreational region by providing people with information about each other's screening results, by providing recreational equipment or accoutrements, by organizing social activities such as dances, games, or contests, and/or by bringing like-minded people together, for example. Information about the mix of people who are already in the recreational facility may be provided to people who are being screened or otherwise deciding whether to proceed toward the recreational region. Within the recreational region in some embodiments, patrons who are screened-but-not-badged or screened-and-badged may mix with patrons who are not medically screened; notice should be given to patrons of the possible adverse medical consequences (e.g., contracting a disease, conceiving an unwanted child) of interacting with a person whose medical status is unknown.

In some embodiments, the invention provides methods for reducing risks for a collection of people within a recreational facility. Some methods comprise medically screening each person in the collection to determine their medical status with respect to at least one of disease status and fertility status, particularly when that status is not readily visible without medical testing; obtaining permission from each person in the collection to share results of that person's medical screening with other people in the collection as a condition of entry to a specified region of the recreational facility; allowing medically screened people from whom permission is obtained to enter the specified region of the recreational facility; and making medical screening results of each person in the specified region of the recreational facility available to the other people in the specified region of the recreational facility.

Some methods further comprise barring a person from the specified region of the recreational facility for at least one of the following reasons: that person has not been medically screened, that person has not given permission to share results of their medical screening with people in the collection. Some methods further comprise refusing to disclose medical screening results to a person for at least one of the following reasons: that person has not been medically screened, that person has not given permission to share results of their medical screening with people in the collection. Some methods donate a portion of facility's revenue to programs for sex education, battered women's shelters, sex abuse treatment programs, and/or other charitable efforts.

The invention may also provide a recreational facility badge which comprises a substrate of durable wearable material; at least one strap extending from the substrate; a medical screening result displayed on the substrate visibly indicating results obtained by medically screening a person with respect to at least one of disease status and fertility status; and a fastening means for fastening the strap to the badge after wrapping the strap about a portion of the person's body, thereby attaching the badge to that person. Anti-counterfeiting components may also be present in the badge, e.g., a holographic logo displayed on the substrate; an anti-theft tag fastened to the substrate; a transponder fastened to the substrate; and/or an encoded checksum or signature printed on the substrate. Other badge components may include a timestamp result displayed on the substrate visibly indicating when the medical screening results were obtained; a bearer identification displayed on the substrate visibly indicating physical identifying characteristics of the person to whom the badge is attached; and/or preference assertions displayed on the substrate visibly indicating preferences of the person to whom the badge is attached. In some alternatives, the badge information (e.g., medical status, preferences, limited identification) is printed directly on the patron's skin.

Other aspects and advantages of the present invention will become more fully apparent through the following description.

### BRIEF DESCRIPTION OF THE DRAWINGS

To illustrate the manner in which the advantages and features of the invention are obtained, a more particular description of the invention will be given with reference to the attached drawings. These drawings only illustrate selected aspects of the invention and thus do not fully determine the invention's scope.

FIG. 1 is a diagram illustrating components and pathways in a secured medically screened recreational facility according to the invention.

FIG. 2 is a diagram similarly illustrating an alternative secured medically screened recreational facility according to the invention.

FIG. 3 is a diagram illustrating another alternative secured medically screened recreational facility according to the invention.

FIG. 4 is a diagram illustrating yet another alternative secured medically screened recreational facility according to the invention.

FIG. 5 is a diagram illustrating a badge of the invention which is produced in part by a medical screening process, and which is suitable for displaying medical screening results and/or other information about patrons in a recreational facility.

FIG. 6 is a diagram illustrating a display of the invention which is suitable for displaying medical screening results and/or other information about patrons in a recreational facility.

FIG. 7 is a flowchart illustrating methods of the invention, from the perspective of medical personnel, security personnel, and/or other personnel in a recreational facility.

FIG. 8 is a flowchart illustrating methods of the invention, from the perspective of a patron (past, present, or potential) of a recreational facility.

FIG. 9 is a diagram illustrating components and pathways in a module in a multilevel secured medically screened recreational facility according to the invention.

FIG. 10 is a diagram illustrating several modules on one level of a secured medically screened recreational facility according to the invention.

FIG. 11 is a flowchart illustrating promotional methods of the invention.

### DETAILED DESCRIPTION OF EMBODIMENTS

In describing the invention, the meaning of several important terms is clarified, so the claims must be read with careful attention to these clarifications. Specific examples are given to illustrate aspects of the invention, but those of skill in the relevant arts will understand that other examples may also fall within the meaning of the terms used, and within the scope of one or more claims. Words used herein do not necessarily have the same meaning they have in everyday usage. Terms may be defined, either explicitly or implicitly, here in the Detailed Description and/or elsewhere in the application file. In particular, several definitions are provided below, and others are provided elsewhere in the specification. It is not necessary for every means identified in a given definition to be present or to be utilized in every embodiment of the invention.

Some Definitions

“Authentication means” comprise tools and techniques for authenticating employees and/or other representatives of a recreational facility, and other authorized persons (e.g., governmental licensing and inspection officials acting in their official capacities). This implies some capability to detect unauthorized persons through a lack of authentication. Suitable authentication means comprise employee identity badges, biometric identification systems, and the recognition of a person as trusted by another person who is already trusted when the trustworthiness of some one or more core employees is a given starting point. Facility owners and managers are considered employees for authentication purposes even if they do not receive salaries.

“Bodily fluids” comprise blood, feces, mucus, saliva, semen (including pre-ejaculate and ejaculate), sperm, urine, and/or vaginal secretions. A transfer of bodily fluids or diseases “between” two people A and B comprises direct transfer only from A to B, only from B to A, or from each of them to the other.

“Demarcation means” comprise items for marking limits of a physical region in a building or other venue. Examples include walls, partitions, curtains, ropes or ribbons, markings on the floor, boundary strips, and/or changes in floor or ground level, e.g., the level change that demarcates a conversation pit.

“Display” may be used in a verb or a noun. In noun forms, “display” refers to badges worn by or printed on people; computer terminals or other computerized device screens; printed wall displays; hand-written surfaces such as chalkboards, whiteboards, spaces on a wall, sheets of glass or plastic; and other items that bear informational indicia. In verb forms, “display” refers to placing informational indicia on a display (noun), altering the informational indicia on a display (noun again), and/or making the informational indicia that is on a display (noun again) perceptible to another person.

“Displaying information indicating” a fact, opinion, or offer means displaying text, images, and/or making sounds which state, depict, and/or convey the fact, opinion, or offer in question.

“Facility goods” comprise goods for use within a recreational facility. The exact goods may vary in different facilities, but examples suitable for at least some facilities include adult toys; books; CDs; condoms, contraceptive sponges, and other prophylactics/contraceptives; DVDs; jewelry; lingerie; lotions; sexual aids; T-shirts and other clothing bearing slogans and/or trademarks; and videos.

“Fastening means” comprise items for fastening a badge's strap around someone to the badge or otherwise help attach the badge to a person. Examples include adhesives, chain, clips, knots, plastic or metal rivets, staples, string, tape, thread, and other fasteners.

“Fertility compatibility” is present between two people when a medical screening shows a low-to-none risk that an unwanted child will be conceived by any sexual acts between those people even if there is a transfer of bodily fluids between those people.

“Include” means comprise unless otherwise indicated. Thus, a statement that X includes A, B, and C means that A, B, and C are instances of X and that there may also be other instances of X in addition to A, B, and C.

“Infertile” or “nonfertile” refers to a person who is medically unlikely to conceive offspring regardless of their sexual partner's fertility status. For purposes of the present invention, examples of infertile persons comprise men with extremely low sperm counts and/or extremely low sperm motility, men who are sterile as the result of a vasectomy, women who are sterile as the result of a hysterectomy, pregnant women (since additional conception is unlikely during the pregnancy), post-menopausal women, women in whom a long-term hormonal or mechanical contraception is established, and persons whose physiology does not include the sperm/egg production and delivery capabilities need for conception.

Note that even though the present invention may be used to reduce the risks of sexual activity involving nonfertile people, and children are generally nonfertile, that most emphatically does not imply any support for sexual activity involving children. The inventor does not condone sex with children, and the invention should not be used to facilitate sex with children.

Note that “fertile” and related terms (infertile, fertility, etc.) are used here in a somewhat different sense than their common usage, e.g., by considering a pregnant woman infertile.

Note also that mere use of contraceptives does not necessarily render a person infertile under this definition, because fertility status can depend on the type of contraceptive. If the only thing preventing conception is a condom, then a man who is physically capable of impregnating a woman is fertile under this definition even if he intends to use, and does use, the condom. People are classified as infertile herein if they are physically unable to conceive, or alternately if the risk of conception is extremely small due to their physical (medical, physiological) condition, e.g. low sperm concentration. An infertile person is one who, as the result of some medical status that is beyond their immediate control or otherwise unlikely to change during their stay within a secured facility, is unable—or at least very unlikely—to impregnate (if male) or conceive (if female).

“Informational indicia” comprise visible, audible, and/or tactile indicia such as colors, letters, icons, symbols, and/or patterns which convey information to one who perceives them.

“Medical screening means” comprise diagnostic tools for screening patrons of a recreational facility to determine their medical status with respect to at least one of two sex-related conditions, namely, sexually transmitted disease status and fertility status. In some embodiments the medical screening means comprise tests for contagious diseases that are not typically transmitted sexually, such as tuberculosis. It is not necessary for every screening means identified here to be present or to be utilized in every embodiment of the invention. In general, the screening means used should give reliable results with regard to the status of sexually transmitted diseases, unlike the visual inspections done in some brothels, for example. The screening means should also provide results in a reasonable time, e.g., less than an hour or two, when the patrons being screened are restricted to prevent intervening activity that renders their screening results inaccurate. However, some embodiments use medical screening means that take longer than that.

Screening may be done by means of medical testing at a secured recreational facility, by means of verification at the secured recreational facility of medical testing or procedures (e.g., surgery) done elsewhere, or by a combination of these two approaches.

Suitable screening means for determining sexually transmitted disease status include so-called rapid tests for chlamydia, gonorrhea, hepatitis, HIV, syphilis, and/or other sexually transmitted diseases, and non-rapid tests for sexually transmitted diseases. In some embodiments, such as those that provide secured separate lodging for patrons who have not yet completed their medical screening, sexually transmitted disease tests that do not provide rapid results may be used as medical screening means.

Suitable screening means for determining fertility status include so-called rapid tests for ovulation cycle status, pregnancy, sperm concentration levels, and sperm motility, for instance, and non-rapid tests for those conditions as well as tests to determine whether a person has been sterilized through a vasectomy or hysterectomy, and whether a person's body contains an implanted long-term contraceptive such as one that regulates hormones or a mechanical device such as an IUD. In some embodiments, such as those that provide secured separate lodging for patrons who have not yet completed their medical screening, fertility tests that do not provide rapid results may be used as medical screening means.

Some rapid fertility tests and some rapid disease tests exist now, and others may be developed and used according to the invention hereafter. It is not necessary to use every test, or test for every disease or fertility condition, in every embodiment. The invention does not claim the medical tests in and of themselves; it is directed to fertility testing and/or disease testing only in combination with other limitations as set forth in the claims.

Suitable screening means for determining fertility status also include means for verifying surgical sterilization and/or mechanical contraception, such as scans using computer-assisted tomography, magnetic resonance imaging, nuclear magnetic resonance, sonograms, x-rays, and the like. Also included are hormone level tests to determine whether long-term hormone-based contraception is in place in a patron.

Suitable screening means for determining fertility status and/or disease status also include physical examination by trained medical personnel, e.g., to determine what organs are present, and to check for visible signs of disease. A medical history may also be taken, and utilized by trained medical personnel in determining fertility status and/or disease status.

Suitable screening means also include means for verifying tests, vaccinations, and/or fertility-related surgery that was done elsewhere e.g., contact with an authenticated hospital or doctor to obtain or confirm medical records. The nature of the screening, e.g., whether it was done in the facility or done elsewhere and confirmed in the facility, may be indicated to other patrons on a badge or other display.

“Physical security means” include structures and/or personnel which separate a secured region from other regions. Suitable physical security means include alarm systems, anti-theft tag detectors, fences, identification authentication systems, locked doors, motion detectors, RF transponder detectors, trained security personnel, and walls.

“Promoting” a process or a thing means marketing, advertising, offering for rent or sale, renting, selling, or otherwise seeking direct or indirect commercial benefit from that process or thing. Promotion will typically be directed at the general public, patrons of a recreational facility, potential patrons, participants in recreational activities, potential participants, investors in businesses that provide a recreational facility and/or related services, and/or potential investors.

“Recreational activity” is activity that refreshes, restores, invigorates, relaxes, diverts, and/or otherwise helps people cope with the stresses of life. For purposes of the present invention, examples of recreational activity comprise viewing or participating in art, contests, conversation, dancing, dining, flirting, foreplay, games, movies, play, sex, socializing, sports, and/or theater.

“Regions” in recreational facilities are physical regions, as opposed to virtual or simulated regions that exist only in a computer. Regions on badges attached to people are also physical, but such badge regions may show information that corresponds to, or is derived from, database records that are stored in a computer.

“Sexually transmitted diseases” are diseases that are transmitted from one person to another solely, or frequently, through sexual activity. The sexual activity resulting in disease transmission does not necessarily involve genital contact in every case with every disease. Other names used for at least some sexually transmitted diseases comprise the acronym “STD”, the term “sexually transmitted infection” and its acronym “STI”, and the term “veneral disease” and its acronym “VD”. For purposes of the present invention, examples of sexually transmitted diseases include: AIDS/HIV, bacterial vaginosis, candida, chlamydia, cystitis, gardnerella, genital warts, gonorrhea, hepatitis, herpes, human papilloma virus, molluscum contagiosum, non-specific urethritis, pelvic inflammatory disease, pubic lice, scabies, syphilis, trichomonal vaginitis, trichomoniasis.

“Status indication quality control means” include means for controlling the quality of medical status indicators (STD status, other contagious disease status, and/or fertility status) to thereby make the indicators more reliable as accurate indications of a person's actual current medical status. Examples include using licensed medical personnel (or using only licensed medical personnel) to perform medical tests during medical screening; using rapid tests in combination with non-rapid tests for the same or overlapping medical conditions; using multiple medical screening means to test for a given medical condition; using anti-counterfeiting means in badges that display indications of medical status; using physical security means to limit access to recreational regions; using trademarks to indicate a level of quality control associated with a particular source of medically screened recreational services; and performing spot checks to verify the validity of badges and/or the current accuracy of badges information displayed on badges attached to people in the recreational region who have been previously medically screened.

“STD compatibility” is present between two people when a medical screening shows a low-to-none risk that any sexual acts between those people will cause either of them to contract an STD they did not already have, even if there is a transfer of bodily fluids between those people. Two or more people who each have no STDs are STD compatible, but so are people who all have the same STD(s) as each other.

Recreational Facilities Generally

With regard to STDs and other contagious diseases, one may receive the disease from someone else during recreational activity with them, even though they have been recently tested and were correctly informed at that time that the test showed no disease. They may be infectious because they contracted the disease after they were tested, but before they finished the recreational activity in question. For instance, even a well-meaning person could be tested for HIV, be told correctly that the test shows no HIV in their system, unknowingly contract HIV thereafter, and then honestly tell another sexual partner that they have recently tested negative for HIV and believe that they do not have HIV. They might even show their later partners a certificate or other official document that confirms their HIV-negative test result, which was accurate at the time but is no longer accurate. Similarly, someone may unknowingly contract a contagious disease other than an STD in between the time they are tested and the time they interact recreationally with someone else, and thereby unintentionally infect the latter person.

Accordingly, the present invention provides secured medically screened recreational facilities. Hospitals are not “recreational facilities” for purposes of the present invention, so long as their primary services are medical rather than recreational, but are instead medical facilities. The recreational aspect of a recreational facility supports recreational activities, which are not necessarily sexual, depending on the embodiment and the circumstances. People patronizing the recreational facility are medically screened for STDs and/or other diseases, and the facility is secured to reduce inadvertent infection of those people. Security may prevent screened people from exiting, becoming infected outside the facility, and then re-entering the facility without being medically tested again. Security may also prevent other people, who have never been tested, from entering the facility and infecting people there. Facility employee—patron interaction may be limited to prevent employees from infecting, and/or being infected by facility patrons. For instance, activities that are likely to transfer bodily fluids between employees and patrons can be prohibited, with enforcement through removal from employment and/or removal from the facility.

In some embodiments, a recreational facility according to the invention includes a security perimeter to inhibit unauthorized entry into a secured region within the facility. A medical screening region operates inside and in conjunction with the security perimeter to inhibit entry of persons whose medical status is unknown (with regard to at least one of the presence of sexually transmitted disease, the presence of other communicable disease, and fertility), and to allow into the secured region persons whose medical status is known as a result of information obtained in the medical screening region. The information may be obtained by medical testing done in the facility, by verification of medical testing done elsewhere, or both.

Depending on the embodiment one is focused on, the facility may operate the medical screening in at least two ways, and in either case may have at least two primary purposes with corresponding equipment, rules, and other infrastructure. The facility may operate to let only people who show no diseases in their screening into the secured region, or it may operate to let in people who have one or more diseases but who are matched with others already having the same condition or a compatible condition, based on medical screening results, so that the number of people with that condition does not increase. The facility may have facilitation of safe and enjoyable sexual activity between consenting adults as a primary purpose, or the facility's purpose may be primarily (or solely) the facilitation of non-sexual recreational activities.

In view of this, one approach that may be taken in a facility according to the invention is to focus on facilitating non-sexual recreational activities among people who have been screened by medical testing and found free of specified communicable diseases. Another possible approach in a facility according to the invention is to focus on facilitating non-sexual recreational activities among people who have been screened by medical testing and organized into groups according to which communicable disease(s) they carry, e.g., a track & field sports facility for people (adults or children) who already have HIV/AIDS. Yet another possible approach in a facility according to the invention is to focus on facilitating consensual sexual recreational activities among adults who have been screened by medical testing and found free of communicable diseases. A fourth possible approach in a facility according to the invention is to focus on facilitating consensual sexual recreational activities among adults who have been screened by medical testing and found to carry disease(s). They may be organized into groups according to which communicable disease(s) they carry, e.g., a private club limited to adults who already have herpes and/or people may be grouped inside the facility according to their fertility, e.g., by not grouping a fertile man with a fertile woman. One facility according to the invention may be marketed and operate as a heterosexual couples “getaway” while other inventive facilities serve gay, lesbian, or bisexual people. A given facility might also serve more than one of these groups. Likewise, a facility might focus on serving HIV+ patrons, HIV− patrons, or both (with suitable notice to patrons), or focus based on other serious diseases. A given facility may operate according to any one or more of these approaches, or other approaches consistent with the claims.

Rather than either grouping people according to their medical status, or allowing only one group of people (as defined by their shared medical status) into the facility, the invention may also be used simply to provide people inside a region with current, reliable, and readily visible information about each other's medical status. The information can be provided via badges worn by people, via data terminals accessible to patrons of the facility, through some other display, and/or by implication via the presence of screened people in secure regions of the facility. People inside the recreational regions can then decide for themselves how (or whether) to interact with other people in view of that information about their own medical status and the evident medical status (or lack of clearly indicated status) of those other people.

The facility 100 may operate with the legal status of a private club, and impose membership restrictions accordingly. In other embodiments, the facility 100 operates with another legal entity status, such as corporation, agency, partnership, city, limited liability company, or otherwise as permitted by law. Operating the facility under the auspices of a focused sovereignty, such as those covering Native American land, UK Channel Islands, federal land, and so forth, or operating partially in international waters, may provide legal and social benefits. An analogy may be drawn, for example, to the operation of gambling establishments. By making the inventive facility 100 subject to the jurisdiction of a focused sovereignty, it may be possible to operate it in ways that advance public health, provide revenue for Native American or other sovereign people, help meet the sexual needs and preferences of a part of society, and/or help reduce the risk of conflict by limiting interaction between that part of society and other parts which disapprove of such sexual activity for personal reasons. Embodiments of the invention can be adapted as necessary to the cultures and laws of various jurisdictions, in a given country and in various countries.

Recreational Facilities in Greater Detail

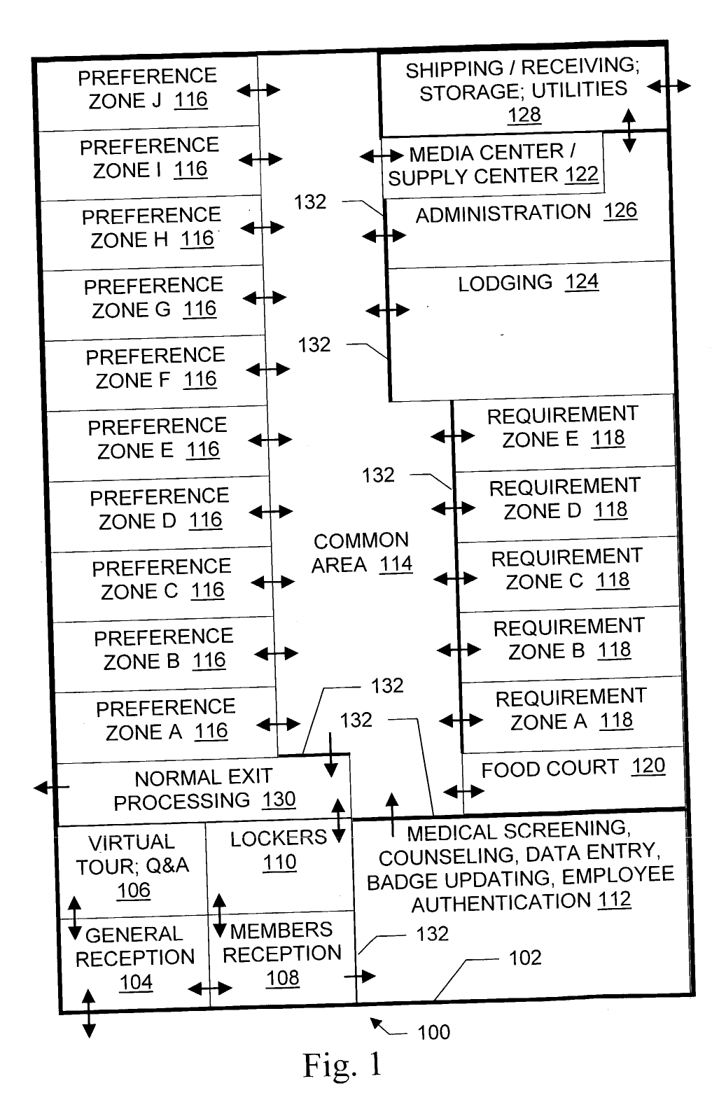

FIG. 1 is a diagram illustrating generally embodiments of a secure medically-screened recreational facility according to the present invention. The diagram is a floor plan, which is provided to illustrate suitable components of a facility, and their associated access paths and security barriers, rather than specifying required region dimensions, absolute region locations, or region counts. Some facility components may be omitted, relocated, supplemented, renamed, grouped, or otherwise varied in some embodiments.

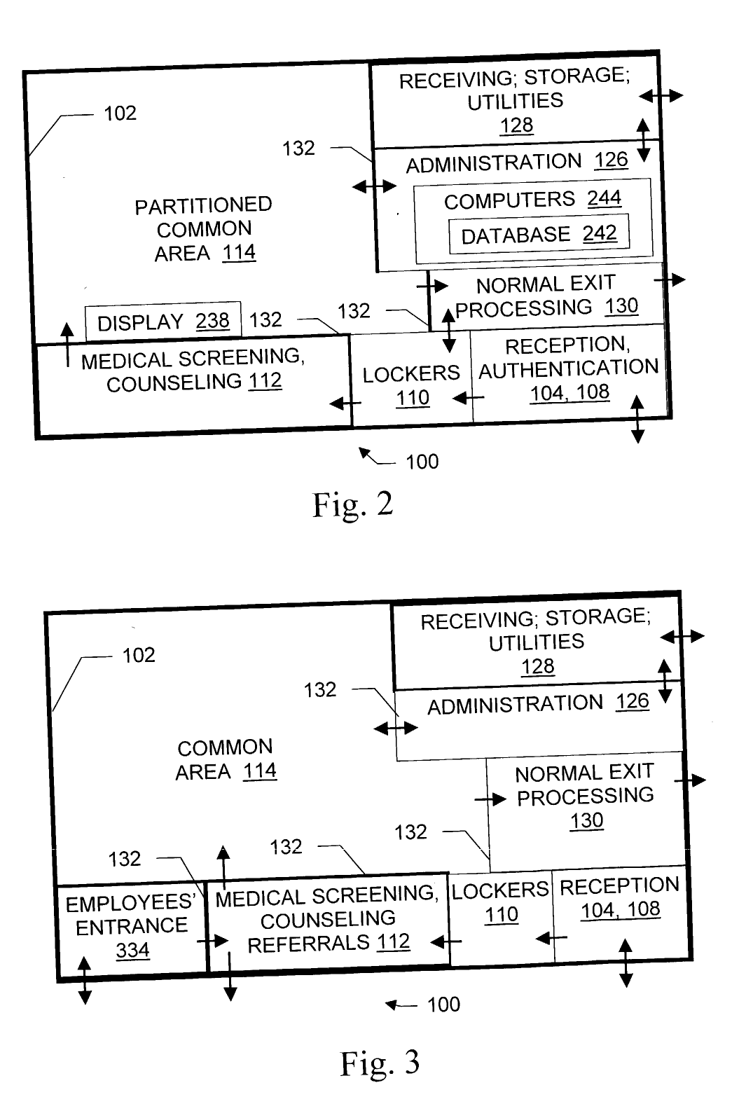

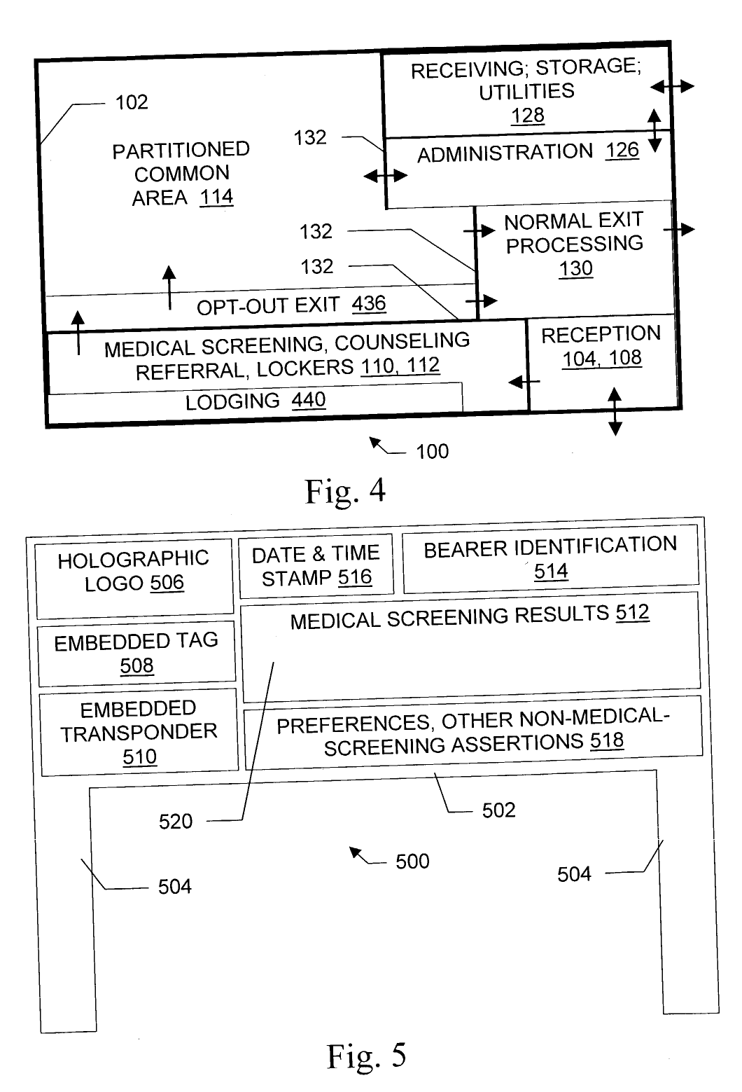

For instance, FIG. 2 is a diagram illustrating other embodiments of a secure medically-screened recreational facility according to the present invention, in which some components and component functionality shown in FIG. 1 have been omitted, some components have been relocated to change the access paths, employee authentication has been relocated to the reception area, and the two reception region components shown in FIG. 1 have been combined. FIGS. 3 and 4 similarly show alternative embodiments to help illustrate the invention. Other embodiments of the invention, not shown in these Figures, are also possible.

The illustrated plans for a secure medically-screened recreational facility 100 show a security perimeter 102 around the facility's interior. The security perimeter 102 uses physical security means to inhibit unauthorized entry into the facility 100. The facility may be embodied in a building, tent or other habitation, or it may be in several habitations, or it may be an open-air but physically secured outdoor environment. A facility may comprise multiple buildings and surrounding roads and grounds, or it may be located in just one building, or in only a portion of one building. It may be on a single level, or on several floors of a building. The facility should have walls, trees, open space, and/or other barriers (and all necessary licenses, permits, etc.) needed to comply with all valid applicable laws, including zoning laws and laws on public decency.

The security perimeter 102 and other security means are said to “inhibit” unauthorized movements rather than “prevent” them, because security measures can be worthwhile even if they are not 100% effective at preventing unauthorized movements in every conceivable situation. In some embodiments the facility's security system can detect an unauthorized entry that was not prevented, and notify facility authorities with a silent or audible alarm to discourage similar future entries.

Normal exits may also serve as emergency exits. For clarity of illustration, other emergency exits are not necessarily shown in the Figures. But it is understood that the facility 100 should comprise suitable emergency exits through the security perimeter 102, with the exits located and operating in compliance with applicable laws and regulations. Likewise, other aspects of the facility 100 and other embodiments of the invention (methods, devices) are assumed. The Figures assume suitable plumbing, heating and ventilation, electrical, and other physical components. The Figures also assume, without specifically depicting, compliance with all zoning, licensing, and other applicable laws and regulations.

More generally, different jurisdictions may impose different legal requirements on embodiments of the present invention. It is the sole responsibility of licensees under this patent and corresponding patents in other countries to identify, and to comply with, all valid and applicable legal requirements in each instance. Nothing stated herein implies or warrants that a particular embodiment will satisfy the legal requirements of a particular jurisdiction, other than the requirements of patent law as presently understood.

In FIG. 1, an optional general reception region 104 is located at the main entrance of the facility. This reception region is for the general public, including those who may be interested in meeting the requirements for admission into the secured portion of the facility. Access further into the facility is initially barred by security means.

More generally, the facility 100 may have various security barriers 132 and entrance restrictions associated with different parts of the facility; the external security perimeter 102 is not necessarily the only operating security perimeter within the facility 100. In addition to physical equipment such as electronically locked doors, security restrictions 102, 132 may be enforced by appropriate facility personnel. Security personnel should be subject to the same limitations on communicable diseases—or, if necessary, protected against such diseases through protective clothing, vaccinations, prohibitions from participation in sexual activity within the facility, etc.—as the patrons in the facility 100, in order to prevent the spread of diseases.

An optional virtual tour region 106 accessible through the general reception region 106 may be provided in the facility to help explain the facility's etiquette, fees, history, hours, layout, legal status, mission statement, philosophy, rules, services, and/or other characteristics of interest to potential patrons and/or to the press or the general public. Live or recorded presentations may be provided, through video or computerized multimedia, for instance, or a live Question-and-Answer session with an authorized facility representative. Depending on the facility's focus, the content of the presentations, and local customs or laws, access to the tour region 106 may be restricted by excluding minors.

An optional member's reception region 108 is available to members. This may be desirable if the facility operates as a private club or other membership organization, for example. Functionality of this region 108 may be provided elsewhere, e.g., FIG. 2 shows a combined reception region 104, 108 which serves both the general public and patrons of the facility 100. People who are medically screened and admitted to a secure region of the facility may be referred to as members or patrons herein. Except as explicitly stated, the facility need not operate as a membership organization even when “member” is used to refer to patrons.

A reception region may include announcements, notices, schedules, and similar information directed at people who will likely be using the secured part of the facility 100. It may be the location at which entrance fees, equipment rental fees, medical testing fees, membership fees, and/or other service fees are collected. Facility coupons may be provided to patrons in this region, to be used for obtaining food or items within the secured region(s) of the facility 100. Facility goods may also be sold, rented, or otherwise provided here.

Legally significant acts may also be performed in reception region 108 and/or elsewhere in the facility 100, or as a prelude to entering the facility 100. Examples include legally significant acts such as: verifying that patrons are not minors; obtaining limited confidentiality waivers to permit sharing of personal information (fertility status, sexually transmitted disease status, other disease status, preferences, and/or other personal information) with others in the facility and prohibit disclosure of that information outside the facility, including sharing such information with other patrons and with authorized employees of the entity that owns/operates the facility 100; obtaining informed consent after giving patrons a notice of the type used, for instance, on adult web sites; and/or obtaining informed consent for medical tests to be done on patrons to determine their fertility status and/or STD or other disease status. It is expected that signatures on confidentiality agreements, informed consent forms for medical testing, informed consent forms for viewing sexually explicit activities, liability waivers for errors or omissions in the medical testing process, and/or other legal documents will be required as a condition of entry to secured regions of some embodiments of the facility 100.

Likewise, it is expected that appropriate steps will be taken to exclude minors when the facility's focus includes activities that are not appropriate for minors. Authentication means and/or identification checks with passports or driver licenses, should be used to verify that legal age requirements are satisfied before allowing a person to enter a secured region in which age-restricted activities such as sexual activity or alcohol consumption occur. Similarly, persons who are severely intoxicated, drugged, or otherwise incapable of giving legal consent should be excluded, and transferred to the custody of local authorities for care as appropriate.

In some embodiments, an employee or a computer terminal (for instance) in the member's region 108 or another region provides patrons with gender/preference/medical status statistics and/or other demographic information about the screened population currently in the secured region(s) of the facility. This may inform patrons of the number of heterosexual men present in the secured recreational area of the facility, for example, or provide similar group demographic or statistical information a member can use to decide whether to continue further into the facility 100 at the present time. Specific patrons may also be identified as present, if they consent to such identification. Information about people who use the facility 100 should be released only with their permission, and only to authorized recipients, using appropriate confidentiality safeguards such as legal confidentiality agreements and/or technical measures such as passwords and encryption.

An optional locker region 110 may be provided in which members can secure their private possessions before entering the main secured portion of the facility 100. Shower and restroom facilities may be provided near lockers. The locker region 110 may be in bidirectional access communication with an exit processing region 130, as shown in FIGS. 1 and 2, to permit those leaving the facility 100 during the normal course of business to retrieve their belongings from the locker. During an emergency, of course, everyone in the building should leave promptly through any available exit, whether it be a normal exit or an exit used only in an emergency.

Alternately or in addition, patrons may give personal possessions to an authorized and trustworthy facility employee for safekeeping during their visit. When this is done, the locker region 110 can be separate from the exit region 130, and a procedure can be used in which personal belongings are in the custody of facility employees during a patron's stay in the secured region. A patron's belongings are then handed back to the patron by an employee as the patron leaves the facility 100.

The facility may impose rules banning certain items from the main secured portion of the facility 100, for safety, health, legal, and/or financial reasons. Banned items may be kept temporarily in a locker, in employee custody, and/or confiscated and turned over to legal authorities, as appropriate. Banned items may comprise one or more of: alcohol, cameras and other recording devices, cash, cell phones, condoms, drugs (prescribed or otherwise), food, pagers, radios, valuable items of personal property, wallets or purses and their contents, weapons (e.g., knives, other edged weapons, guns, chemical sprays), wireless email-capable devices, and other devices that may compromise the privacy of activities occurring within the facility by transmitting contemporaneous descriptions of those activities to a device outside the facility. In facilities 100 that promote appropriate sexual activities, condoms from outside the facility 100 may be banned, for instance, in order to ensure the quality of condoms used within the facility. In some facilities, cash, checks, credit cards, jewelry, purses, wallets, and other portable valuables may be banned to inhibit their theft and/or to inhibit unlawful prostitution within the facility 100.

A medical screening region 112 serves as a filter to screen in and/or screen out individuals for entry to the main secured portion of the facility and/or for grouping therein. Access restrictions are enforced on patrons, and possibly on facility employees, by physical security means.

Although medical screening and employee authentication each limit access to secured portions of the facility 100, medical screening does so in response to medical status while authentication limits access in response to personal identity and/or role (e.g., employee, health inspector). In some embodiments, the employees of the facility are authenticated by authentication means in the medical screening region 112 before they are allowed to enter the secured recreational region. In some embodiments the employees are authenticated in another region, e.g., reception region 104, 108 in FIG. 2, or employee entrance region 334 in FIG. 3. In jurisdictions that limit or prohibit the participation of entity employees in recreational activities, it may be unnecessary for facility employees to be medically tested, but their identities and authority should be authenticated regardless, to prevent entry of unauthorized persons. The screening, authentication, and physical security means coordinate to inhibit facility access by unauthorized persons, such as persons who should have been tested but were not, or persons who intend to violate patron confidentiality.

Medical testing may be performed within the screening region 112, or the testing may be done elsewhere and the results scrutinized within the screening region 112 for reliability. Medical tests are done to reach medically sound conclusions about one or more of the following, for instance: the presence/absence of sexually transmitted diseases and/or other communicable diseases; fertility status, e.g., whether women are pregnant, whether women are post-menopausal, and whether a person has been sterilized by vasectomy or hysterectomy; and whether a hormonal contraceptive or a mechanical contraceptive is in use.

In facilities 100 that test for serious illness such as HIV/AIDS, and/or test fertility status, counseling services may be provided in the medical screening region 112 to assist people who are learning for the first time that their medical status is substantially different than they believed it was. Counseling may be done in a physical examination room, in a counseling office in the facility, or at another location by a counselor to whom the patron is referred by facility personnel.

Although screening region 112 is denoted in the Figures by a rectangular shapes, it may take other shapes. The screening region 112 may also be subdivided into separate counseling rooms, examination rooms, laboratories, medical test storage rooms, a server room for computers, and so forth, to carry out the methods of the invention and provide badges or displays according to the invention. Each such room should have appropriate furnishings and built-in structures, e.g., physical examination and testing supplies in the examination room (e.g., exam table, gloves, lighting, sink) in the exam rooms, chairs and desk in the counseling room, and so on.

More generally, even though particular embodiments and uses of the present invention are expressly illustrated and described individually herein, it will be appreciated that discussion of one type of embodiment and its uses also generally extends to other embodiment types and their uses. For instance, the description of the invention's methods also helps describe the structures and operation of the invention's systems and devices, and vice versa.

Continuing with FIG. 1, the illustrated embodiment of a facility 100 includes regions identified as preference zones 116. These regions 116 are demarcated by demarcation means to mark their physical limits in a building 100 or other venue 100. Preference zones 116 are not required in every embodiment of a facility 100. A given embodiment may have more or fewer preference zones 116 than shown in FIG. 1, and the zones 116 if present may be sized, shaped, and/or located differently than shown in FIG. 1.

Preference zones 116 allow patrons of the facility 100 to indicate their preference for particular activities by entering a clearly demarcated region. In a facility 100 that allows sexual activity between consenting adults, the preferences may be sexual preferences, e.g., a preference for certain sexual acts, a preference for a certain type of sexual partner, and/or a preference for certain adult toys, costumes, furnishings, or other accoutrements. Each sexual preference zone 116 may be suitably equipped with specialized furnishings (mirrors, pillows, windows, etc.), devices (adult toys, costumes), media tools (computers, magazines, movies, music, etc.), and/or other items and settings that facilitate or enhance sexual activity for consenting adults.

Regardless of whether a facility 100 allows sexual activity, preferences may exist for certain types of social activity (e.g., contests, dancing, dining, games), certain types of music, a certain age range among the participants, or any of the other criteria which people consider when making decisions about social interaction and/or recreational activities. The preference zones 116 help like-minded people to gather and pursue in person their shared interests, by delimiting regions specifically set aside for recreational activities that correspond to those shared interests.

FIG. 1 also illustrates requirement zones 118. Although requirement zones 118 are shown in FIG. 1 with preference zones 116, either type of zone, or both, or neither, may be present in a particular facility 100. Requirement zones 118 may be defined to reflect the same kind of criteria used in establishing preference zones 116. But access to a requirement zone 118 is controlled by security means 132 and entrance criteria to help ensure that the participants meet minimal requirements for entry. By contrast, limits on participation in a preference zone 116 are suggested but are not enforced by security barriers within the facility 100. For instance, a person who has a stated aversion (a negative preference) for viewing or participating in bisexual activity will not necessarily be barred from a bisexual preference zone, but that person should be denied entry to a bisexual requirement zone 118.

Moreover, security measures 132 for requirement zones 118 in some embodiments enforce the compatibility of medical screening results of those who are admitted to the zones 118. For instance, an HIV-positive requirement zone 118 should bar entry to persons who are not HIV-positive, and an HIV-free requirement zone 118 should bar entry to persons who are HIV-positive. Aside from the additional security barriers, requirement zones 118 may be demarcated, like preference zones 116, by using demarcation means.

FIG. 1 shows a food court 120, which may provide food and drink to patrons. The food court contains tables or counters, chairs or stools, and food service equipment. Under appropriate licensing and with suitable safety precautions, alcohol may also be provided. In facilities 100 which ban cash, checks, and credit/debit cards from the secured regions, facility coupons may be used by patrons to obtain food and drink, or the food and drink may be included in the price of admission to the facility 100.

A media/supply center 122 provides patrons with facility goods for use within the secured regions 114, 116, 118. These may be complementary, or facility coupons may be used by patrons to obtain them. Facility goods are not sold or rented in a secured region 122 in embodiments which ban cash from the secured regions, unless provision is made for payment outside the secured region.

Some facilities 100 include a lodging region 124, similar to hotel lodging but subject to the facility's security and medical screening measures. For instance, in facilities that let only medically screened people into a secured recreational region, if the lodging region 124 is inside that secured recreational region then only medically screened people should be allowed into the lodging region 124. Except in an emergency, the patrons should be able to leave the facility only by way of the exit processing region 130. Likewise, if the only people allowed in a recreational region are medically screened people and employees who are not necessarily medically screened but forbidden from activities that could spread a disease screened for, then only people meeting that description should be permitted in a lodging region 124 within that recreational region. FIG. 4 shows a lodging region 440 which is located in the medical screening region, to lodge patrons who are not yet fully screened, such as patrons awaiting the results of lab tests that are not of the “rapid test” variety. As with the other figures, the diagram in FIG. 4 is not necessarily to scale. Regardless of their location, lodging regions 124, 440 may provide amenities such as billiards, DVD/video viewing equipment, hot tubs, private rooms, showers, and other amenities. Such amenities may also be provided in recreational regions 114 that lack a lodging region.

To provide an assurance that the medical screening results for a given person continue to be accurate after the screening for as long as the person is free to interact with other people in the facility 100, the facility 100 should prevent unscreened entry to its secured regions from outside the security perimeter. For instance, if a test for sexually transmitted or other contagious diseases is negative, one can be reasonably certain that no such disease has been contracted since the test if the tested person has not interacted since the test at close range with anyone carrying the disease. Accordingly, if a person leaves the facility 100 and then wishes to re-enter it, they should be medically screened again, to detect any infections that may have occurred while they were outside the facility. Some patrons may wish to avoid the re-screening effort and expense; lodging within the security perimeter may permit them to so that. In some cases, a facility 100 may nonetheless have rules requiring re-screening every stated time period (e.g., every three days) even when the person to be screened has apparently been continually lodged within the facility. A facility may also conduct random spot checks to retest people independently of when their last test occurred.

When the facility 100 is embodied in a building, it will often have a region 128 for deliveries, heating/air conditioning equipment, item storage, and other supporting infrastructures. Suitable security measures should be taken to prevent unscreened persons from surreptitiously entering secured regions of the facility 100 through such regions 128.

In the exit processing region 130, patrons should retrieve (or have facility employees retrieve for them) personal belongings left in the lockers 110 or left with a facility belongings custodian. Also, any badges worn by the exiting patron should be removed, void-stamped, or otherwise invalidated to ensure that re-screening occurs before the patron is permitted back into secured regions 114, 116, 118. In the exit region 130, patrons may also be notified of upcoming events, or reminded of facility rules and etiquette. Some methods of the invention include setting rules of etiquette for sexual activity performed within the secured portion of the facility, and some further include the step of enforcing the rules by ejecting and/or denying entrance to persons who violate the rules. Sexual activity includes flirting, foreplay, and/or observation of others engaging in such activities, as well as actions involving contact or other stimulation of sexual organs.

As noted, FIG. 3 shows an employee entrance region 334 which includes authentication means for verifying the identity and authority of facility employees. Such a separate entrance allows authorized employees of the facility to reach the medical screening region 112 (or the common area 114 directly from region 334, an option not shown in this Figure) without going through the path used by patrons of the facility.

Another option indicated in FIG. 3 is the use of referrals to counselors located outside the facility, instead of providing counseling in the facility 100. Other embodiments of the facility allow neither for counseling therein nor referrals elsewhere.

Another option indicated in FIG. 3 is the presence of an exit directly from the medical screening region out through the security perimeter 102. This option allows patrons to leave from the medical screening area 112 without going through the common area 114. This may be beneficial if medical test results lead patrons to change their mind about entering, or are such that the facility denies them entry to the recreational area 114. It may also ease handling of patrons who behave in an unruly or aggressive manner.

FIG. 4 illustrates another optional component of the facility 100, namely, an opt-out exit region 436. This region 436 is on the secured path after the medical screening region 112. Patrons may go from the medical screening region directly to the recreational area 114, although that option is not shown on this Figure. Alternately, patrons may be directed to the opt-out exit region 436 where they are given access there to real-time views of activities presently occurring in the recreational region 114. Such viewing should be done only with patron permission. Views may be provided by windows, one-way mirrors, or video feeds to monitors in region 436, for instance. Patrons in the opt-out region 436 can then decide to continue into the recreational region 114, or they can opt out by going directly to the exit processing region 130 and leaving the facility 100.

The following example scenarios are provided to further illustrate various embodiments of the invention. In one scenario, a member of the general public enters the reception region 104, then attends a presentation in the Q&A region 106, enrolls as a club member, enters the members' reception are 108, leaves personal belongings in the locker room 110, is medically screened and receives a badge 500 in the screening area 112, is permitted to enter the secured recreational area 114, engages there in recreational activities, visits the food court 120, then leaves through the normal exit processing region 130. In another scenario, a person enters a combined reception area 104,108, attempts to enter a Q&A region 106, is barred as a minor, and is then escorted out of the facility 100 without gaining access to regions reserved solely for adults. Alternately, one might enter the reception area 104, be screened in the screening area 112, learn there that one is not fertile, receive some counseling, and then decide to leave the facility 100 instead of continuing into the common area 114. As another example, an employee enters the authentication region 334, is authenticated there, continues through the medical screening area immediately (without being medically screened at this time) into the common area 114 to the administration area, goes back to the common area 114, and eventually leaves by way of the normal exit processing region 130. It will be understood that many other scenarios are also possible.

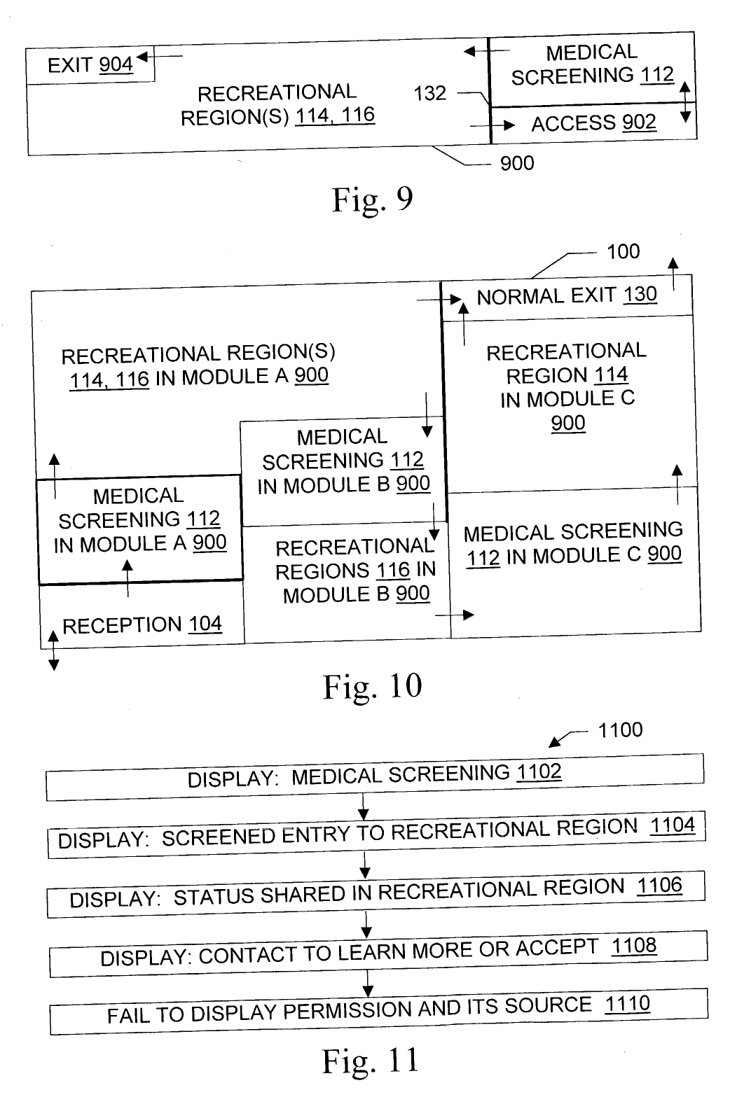

Some embodiments of a recreational facility according to the invention are arranged in successive rings, layers, or levels, for instance. For brevity, such a ring, layer, level, or other successive secured and medically screened recreational portion of a facility is termed a “module”. Modules 900 are illustrated in FIGS. 9 and 10; other modules are also provided by the invention. Each module 900 has its own medical screening region 112 or portion thereof, and also has physical security means. The medical screening and security operate in combination to determine the status of people and control their access to a secured recreational area of that module 900. The criteria for admission to secured recreational areas follows an order or progression as a patron travels through successive modules 900. Those who do not meet the admission criteria can exit the facility through a normal exit region 130 of the module, or if they have already been admitted to at least one recreational region they can return to such a region.

FIG. 9 illustrates a one-level-module 900 of a multilevel facility 100. Entry to the module 900 is normally secured so patrons enter through access region 902, which may include stairs or an elevator, for example. Patrons wishing to enter this module's recreational region must pass the module's medical screening area 112, where they are tested or otherwise medically screened. Those who meet the admission criteria are allowed to enter the recreational region; those who do not are directed back through access 902 to a previous module or the facility's exit. People who have been admitted to the module's recreational region may leave it by an exit 904 that exits the facility 100 without access to other modules 900, or they may go back through access 902 to a previous module or the facility's exit. Other modules 900 on other levels of the illustrated facility 100 have a similar configuration and manner of operation.

FIG. 10 illustrates three modules, denoted A, B, and C, on one level of a multi-module facility 100. As in the other Figures, arrows indicate directions of travel allowed by the physical security means during normal operation of the facility, and the facility contains emergency exits as called for by applicable regulations, regardless of whether those exits are expressly shown in the Figure. FIG. 10 also illustrates that the recreational regions of different modules may have different configurations, such as by including or omitting components shown in the various Figures. In FIG. 10, for instance, the recreational region of module B includes preference zones 116, but the recreations region of module C does not.

In another embodiment, a first module 900 recreational region contains patrons who have been determined by medical screening to be free of gonorrhea; a second module 900 recreational region contains patrons who have been additionally determined by medical screening to be free of chlamydia and syphilis as well as gonorrhea; and a third module 900 recreational region contains patrons who have been additionally determined by medical screening to be collectively infertile (e.g., the third module recreational region contains at most fertile patrons of only one gender). In this embodiment, the risk of contracting a disease that is posed to a person lacking that disease decreases as the person passes into successive module recreational regions, and the risk of being involved in an unexpected pregnancy decreases on entry to the third module's recreational region.

In another embodiment, the risk of transmitting one's disease to another person who did not previously have it decreases in successive modules 900, as only those with a specified STD are allowed to enter a given module's recreational area. However, rather than using successive modules as filters, a facility intended to serve diseased people could also place them in groups using a facility like the one shown in FIG. 1, by performing several tests on them (in sequence or at once) and then providing a separate recreational region for each group according to the test results. For example, requirement zone A could be reserved for people who test 710 positive for gonorrhea and syphilis and negative for HIV, chlamydia, hepatitis, and herpes; requirement zone B could be reserved for people who test positive for gonorrhea, syphilis, and HIV and test negative for chlamydia, hepatitis, and herpes; requirement zone C could be reserved for people who test positive for gonorrhea and test negative for syphilis, HIV, chlamydia, hepatitis, and herpes; and requirement zone D could be reserved for people who test positive for gonorrhea, syphilis, HIV, chlamydia, hepatitis, and herpes. The people in each requirement zone 118 could then interact sexually with one another without spreading any of these diseases to someone who was previously uninfected.

Recreational Facility Databases

A secure database 242 of patron medical status information may be maintained in one or more computers 244 by facility personnel, with privacy safeguards and patron permission. Suitable privacy safeguards include, for instance, encryption of the database 242, legal confidentiality-protecting agreements signed by facility employees, passwords, and physical security means to inhibit unauthorized physical access to computers 244. Computers in the facility may be networked with each other, but the computers 244 that hold the database 242 and/or other confidential information (e.g., billing records, membership records) should have no connection to an outside network such as the Internet.

The facility may have computer terminals or other displays 238 that are visible outside the administration area 126 to make information such as medical screening results and/or patron demographic information available to patrons in reception 108 and/or recreational 114 regions. The ability to change the information visible on a display should be limited to facility medical personnel and other authorized persons. The database 242 may also be maintained on paper or other non-computerized form, either as a supplement to a computerized database, or as a substitute.

The database 242 contains data records that are updated in conjunction with the medical screening process. In some facilities 100, patrons within the secured portion of the facility can access part of the database 242 using computer terminals or other access devices to verify the identity and medical status of another person there before deciding whether to interact with that person. This can help provide quality control to assure that medical status information is accurate. Alternately, the data record content may be copied in a badge carried or worn by the person, which can then be read without a networked computer, and possibly without any computer at all. Both terminals and badges may also be used.

Regardless of how it is recorded and read, the database 242 data record for a given person may provide a reader with information such as the person's identification photo, their personal statistics (age or age range such as “20-29 years old”, gender(s), height, race, weight), as well as one or more of the following: sexually transmitted disease test dates and results for the person, other contagious disease test dates and results, fertility test dates and results, an indication of whether the person in question is known to have left the secured part of the facility since the last round of tests, the person's stated marital status, the person's stated sexual orientation, and the person's stated sexual preferences. Note that in order to help protect patron privacy, identification information commonly found in many databases is not present in a database 242 data record according to the invention accessible to other patrons, namely, the person's name, address, phone number, email and other contact information, and the person's profession.

Recreational Facility Badges

Within the screening region 112, a badge produced in part by the medical screening process may be provided to the screened person. FIG. 5 shows one embodiment of a badge 500 according to the present invention; other embodiments will be apparent to those of skill based on the teachings herein.

Badges 500 need not be used in every embodiment. Some embodiments will admit to the secured region only persons who have “compatible” medical screening results, so no badge is needed to display the screening results. That is, if a person's screening results were not compatible with the screening results for other people in a secured recreational region 114 then the person would not be admitted to that region, so the status of people in a region is made known by their presence in that region.

But other embodiments admit to a recreational region screened people who have with incompatible screening results (e.g., different STDs), and some embodiments allow a mixture of screened and unscreened people in a recreational region. In these cases, badges 500 may be particularly helpful in assessing the risks of interacting with another person. It is expected that badges 500 will be used primarily when some possible interactions of some people are compatible but other possible interactions are not compatible, and/or when the people bearing the badges do so in order to display their non-medical-screening attributes, such as sexual preference or marital status. But badges 500 can be used as visual confirmation and reassurance, and as quality controls, even when medical screening and physical security limit people in the secured regions to those that are compatible regardless of how they interact.

The badge 500 allows other people within the region to ascertain and/or verify the medical status of the person who wears the badge. The badge 500 may also contain assertions which are not the result of medical testing, such as an assertion of the person's marital status and/or an assertion of the person's sexual preferences. Sexual preferences may be made known to others in the secured regions 114/116/118 by a badge, by presence in a designated preference zone 116 or a requirements zone 118, by acting in a manner consistent with the preference, and/or by stating the preference aloud. In some embodiments (regardless of whether badges are used) preferences can be: positive, meaning that the person favors or enjoys X; neutral, meaning that the person does not care one way or the other regarding X; or negative, meaning that the person is averse to X. In other embodiments, only positive preferences are indicated on badges or displays.

Several characteristics are desirable in a badge according to the present invention. It should permit vigorous physical activity, including vigorous sexual activity in appropriate circumstances in some embodiments. In many embodiments it should be easy to read the badge at a distance of at least one meter (approximately three feet), but in other cases the badge face is smaller so it can be easily read only at a distance closer than one meter. In some embodiments the information the badge bears may be made visible to others only on their request, e.g., by lifting a cover flap, and/or made visible to selected other people only at the option of the person the badge pertains to. The badge should be tamper-evident, that is, it should not only be difficult to tamper with the information displayed on the badge, and difficult to transfer the badge from one person to another, but in addition it should be difficult to do so without making it clear to others that such tampering has occurred. The badge should be non-reusable—a new badge is issued as the person clears the screening region 112, and the badge may be removed (or hidden if printed on a person's skin) during normal exit processing 130. The badge should be cost-effective. It should be difficult to counterfeit. Finally, it should be firmly attached or otherwise linked to the person whose information it displays. However, in connection with badges, as with other aspects of the present invention described herein, the term “should” merely denotes desirable characteristics, not characteristics (or features, steps, or structures) that are mandatory in every embodiment. The invention is defined by the claims.

The badge is linked to the person whose information it bears. Suitable linkage may be performed by providing a current identification photo in the badge; by physically attaching the badge to the person using a bracelet, necklace, or anklet; by adhering a badge substrate to the person's skin; by surgically implanting a radiofrequency or similar encoded transponder in the person; by providing the same identifying code on the badge as on the person; and/or by printing the badge information on the person's skin, for example. Any means that reliably ties (a) the badge's informational indicia, to (b) the person those indicia describe, can be used. Printing on the person may be done in a manner similar to that done by amusement parks when they stamp “PAID” or a symbol on the back of one's hand. But in the present case the printing provides different information (medical screening results) in a different context (secured medically-screened recreational facility) for different reasons (to advance recreational activity without spreading disease and without conceiving unwanted children) than has been done in amusement parks. Printing may comprise a bar code for enhanced badge security and quality control.

Facility rules and facility etiquette may state that one is expected to make the badge's information readily visible to others in the secured region, without prompting, as a courtesy. The badge may aid discovery of the badge wearer's information within the secured region by providing the information using indicia that are readily visible from a meter away, such as large letters in a block font (e.g., “F” for fertile; “H+” for “HIV-positive”), color codes (e.g., a green region for “no sexually transmitted diseases detected”, a white region for heterosexual, a purple region for homosexual, and a checkerboard region for bisexual), icons, geometric patterns, and so forth. Some embodiments use the letters and abbreviations commonly found in the personal ads in newspapers or on web sites, e.g., “DDF” for “disease- and drug-free”. Badges 500 may display information that is illuminated and/or use glow-in-the-dark inks.

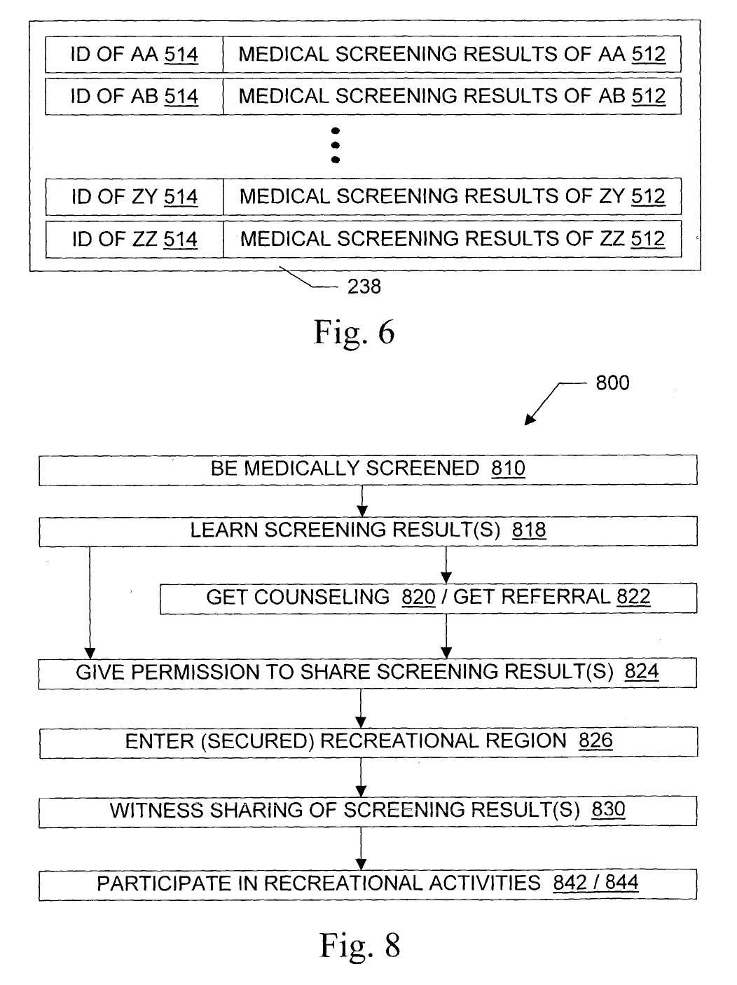

As an alternative to badges, or a supplement to them, a facility may provide one or more “displays” such as display 238 noted in FIG. 2. One embodiment of a display 238 is further illustrated in FIG. 6. The display 238 can be seen by patrons in region 114. Access to the display 238, or to the input device that is used to specify what is shown on the display 238, is controlled by physical security means and authentication means, so the information displayed can be changed only by medical personnel of the facility 100.

The display 238 displays information similar to that shown on badges, such as limited identifying information 514 and medical screening results 512. Although the display shown in FIG. 6 provides only an ID and screening results, some embodiments also display preference information. The display 238, unlike a badge, is not physically attached to a particular person whose information it bears, so the display 238 contains limited identifying information 514 such as a photo or an ID number to provide linkage between that person and the displayed medical screening results 512. Some embodiments display an ID photo as identifying information 514, plus the medical status 512. Some embodiments display an ID number-letter combo 514, plus the medical status 512, and the corresponding ID number-letter combo for a given person is printed on a badge attached to that person (badges may be printed directly on the skin). The ID number-letter combo may include letters only, numeric digits only, other symbols, or some combination of such ID components. The ID number-letter combo should be hard to modify, e.g., avoid digits 3 and 8 which can be too easily transformed into one another, and provide a clear upward orientation to prevent confusion between digit 6 and digit 9. The identifying information on badges 500 and displays 238 is limited to protect patron privacy by not displaying information such as a person's full name, their home address, their work address, their telephone number, or their email address.

In some embodiments, a first person compares limited identifying information 514 with the physical characteristics of second person and/or with an ID number-letter combo printed on or otherwise attached to the second person. This is done to provide the first person with a basis for deciding whether the identifying information 514 identifies the second person, and hence to help the first person decide whether the medical screening results corresponding to that identifying information 514 accurately describe the medical status of the second person.

FIG. 5 shows one of the many possible embodiments of a badge 500 according to the present invention. Other badge 500 embodiments according to the invention are also possible. The illustrated badge 500 shows multiple components that may be omitted from other embodiments; for instance, in some alternatives the badge is formed by printing information (e.g., medical status 512, preferences 518, and/or limited identification 514) directly on the patron's skin, in which case embedded components 508, 510 are unlikely to be used. Illustrated components may also be placed differently. In addition, the illustration is not necessarily to scale.

The illustrated badge 500 has a substrate 502 of durable but wearable material, such as rip-resistant nylon or other cloth, vinyl, or spun-bonded olefin (e.g., TYVEK brand spun-bonded olefin material; TYVEK is a registered mark of E.I. du Pont de Nemours & Co.). One or more straps 504 extend from, and are bonded to (or integral extensions of) the substrate 502. As part of the medical screening process, medical STD/fertility test results and/or other information pertaining to a person as discussed herein are imprinted or encoded in the badge 500. Then the badge 500 is attached to that person's arm, leg, or torso by wrapping the straps around the body and then adhering them to the substrate 502. The adhesion, straps, and substrate should operate in a manner similar to tamper-evident envelopes to make tampering with the badge 500 readily visible.

To discourage counterfeiting, the badge 500 may include one or more of: a logo such as a holographic logo 506; an embedded tag 508 such as an anti-theft tag used in retail stores to prevent theft of books or CDs; and an embedded transponder 510 such as the RF transponder used in identification systems. Other anti-counterfeiting measures, such as a bar code with embedded checksum and/or measures used with currency, can also be employed. If a holographic logo 506 is used, counterfeit badges may be detected when they use logos that are non-holographic, less intricately printed, and/or the wrong color (colors may be changed daily in a random pattern). If an embedded tag 508 is used, counterfeit badges may be detected when they fail to respond to electromagnetic detectors in the same manner as a valid tag. Patrons can be checked for tags or transponders before the badge is attached (they should have none at that point) and then checked randomly or systematically within the secured region 114 (each should have one at that point).

As noted, the badge 500 may be printed with letters, colors, and/or icons indicating the results of the medical screening acts performed on the person wearing the badge; this information appears in a medical screening results region 512 of the badge. The badge 500 may also show the person's testing history, e.g., test dates and test results stretching back in time for months or years. This region 512, and other badge regions, may be covered by a clear plastic laminate 520 which is difficult to write on, and whose removal is readily detected, to prevent tampering with the information placed on the badge by facility 100 personnel in the medical screening region 112 and/or to make tampering evident. A bearer identification region 514 contains information with which a reader can confirm that the badge is on the correct person, e.g., the person's physical description and an identification photo. The badge 500, like other displays of medical status, serves as proof of medical status. It should be provided in non-discriminatory manner, without improper regard to characteristics other than medical status, by a disinterested third party such as medical personnel or other facility personnel.

A date and time stamp region 516 notes the approximate time at which the medical screening was completed, so that readers can verify that the badge wearer has been recently tested. In some embodiments, the physical security, screening, and exit procedures make it unlikely that someone who was recently tested has been in contact with someone else who could substantially change the badge wearer's medical status, by infecting them or by making them pregnant, for instance. The date and time the badge wearer last passed through the normal exit region 130 may also be shown on the badge.

Sexual preferences and other non-medical screening assertions may be shown in another region 518 of the badge. These assertions may be without warranty by the facility management, or steps may be taken to test their accuracy, e.g., by accepting reports from patrons of statements and/or actions that are inconsistent with a particular assertion.

The badge and other medical status displays such as display 238, help people know their health risks, so that they can consider those known risks when deciding whether to interact with another person in the facility 100. If they choose to interact, they can choose to interact in a way that is less risky to others and/or to themselves, e.g., by avoiding transfer of bodily fluids, or by choosing to interact with someone who already has a given STD, or with someone who is infertile. Likewise, the medical screening and the security provisions together assist in risk evaluation and thus help people avoid risky behaviors. Medical screening means identify risks associated with individuals, and security means inhibit entry into the secure regions by individuals whose risks have not been medically determined and identified to others, as well as inhibiting false statements to others regarding those risks.

Recreational Facility Methods

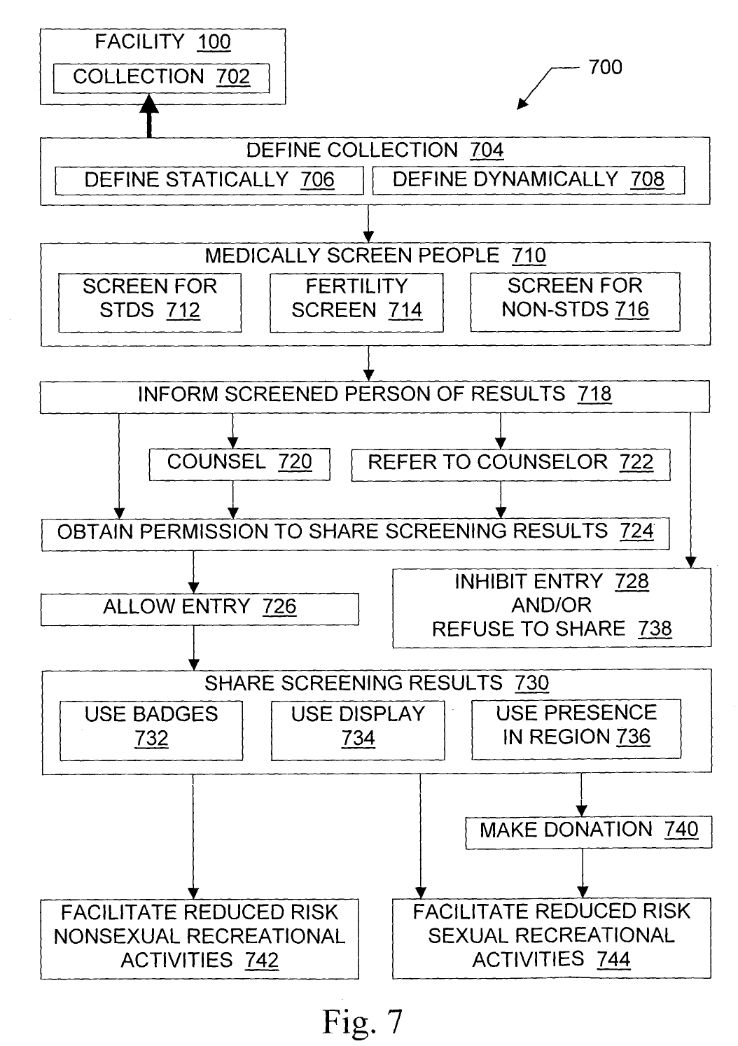

Some embodiments of the invention provide a method 700 for reducing risks for a collection 702 of people within a recreational facility 100, such as the methods illustrated by FIG. 7. During a defining step 704, the scope of the collection may be statically defined 706, such as by listing the people or by listing an alphanumeric identifier for each person in the collection 702. In one alternative, the collection 702 scope is dynamically defined 708 by adding a person to the collection 702 when it becomes clear that person will be subject to all required steps of the method 700, including medical screening 710 and sharing 730 of the screening results. More generally, the collection may be implicitly defined by other steps described herein.

During one or more instances of a medical screening step 710, each person in a collection of people is medically screened. That is, people may be screened one at a time, more than one at a time, or even all at once. The medical screening step 710 can be performed by using medical screening means to determine the person's medical status with respect to at least one of two sex-related conditions, namely, screening 712 for sexually transmitted disease status and/or screening 714 for fertility status. Checks for other diseases than STDs may also be performed 716.

Screening 710 may be performed in order of decreasing seriousness of the condition being screened, e.g., by screening first for life-threatening diseases such as HIV, then optionally continuing by screening for debilitating diseases like syphilis that are not life-threatening or at least need not be life-threatening if diagnosed and treated early enough, and then optionally continuing to diseases like scabies that are typically not life-threatening. Other testing sequences may also be used. Medical tests to determine one's disease and/or fertility status could be sequenced according to the seriousness of the condition tested for, cost, time required to obtain results, relevance to the current population in the facility recreational region as discussed herein, and/or other criteria.

In addition, during a screening step 710 medical personnel may verify that a patron has been vaccinated against one or more contagious diseases (STDs and/or otherwise) and/or they may provide a patron with such vaccination(s). Medical testing may be used to accomplish the screening, as discussed in connection with facilities 100 and/or badges 500 above. Medical testing may be skipped when the medical condition being checked is readily visible without medical testing, e.g., a chemical analysis-based fertility test might not be necessary to determine that a woman is pregnant. The person being screened is informed 718 of their screening results. They may then receive 720 counseling and/or receive 722 a counseling referral.

In some embodiments the medical test(s) done during screening 710 are administered on the facility premises. The person being screened waits in a controlled area without leaving the facility (but without entering the secured recreational portion of the facility) while test specimens are processed to obtain the test results, so that the test results are not inaccurate due to an infection of the person that could otherwise occur between the time the test is given and the time the results are obtained. Such a controlled area 112 may include entertainment and/or lodging 440, for tests that take more than a few minutes to process. Methods of the invention may also require retesting the person each time they leave the secured facility and then want to come back in.

During one or more instances of a permission obtaining step 724, permission is obtained from each person in the collection 702 to share at least some results of that person's medical screening 710 with at least other people in the collection. For instance, if the method screens 712 for STDs, then permission to share the person's STD status can be required as a condition of entry to a specified region of the recreational facility, e.g., secured recreational region 114, or a region that is not secured. A region that is not secured may be provided, for instance, if badges 500 and/or a display 238 are used to alert people in the region to medical status of screened people, and both screened and non-screened people are allowed into the region. Likewise, if the method screens 714 to determine fertility status, then permission to share the person's fertility status with other patrons can be required as a condition of entry to a recreational region, whether that recreational region is secured or not.

During an allowing step 726, medically screened people from whom permission has been obtained 724 are allowed to enter the recreational region. This may be accomplished, for example, by suitable operation of physical security means that previously barred 728 their entry to a secured region 114. Or it may be done without such physical security if screened and unscreened people are allowed to mix, relying on badges or displays to assess their interaction risks instead of relying solely on another person's presence in a secured region to indicate that person's medical status.

During a screening results sharing step 730, the method makes medical screening results (at least) of one or more person(s) in the specified region of the recreational facility available to other people in the specified region of the recreational facility. This may be done explicitly by using 732 badges 500 that are linked to people who have been screened 710 and have given 724 their permission for their screening results to be shared. Sharing 730 may also be done explicitly by displaying 734 medical status on a display such as display 238. The sharing step 730 may also be done implicitly by using 736 a person's presence in a secured region to imply their medical status. For example, a method may share status by letting 726 people enter a secured region that is reserved for people who have a particular medical status, e.g., a requirement zone 118 reserved for HIV-positive patrons, or a secured common area 114 which is fully dedicated to patrons who tested negative for specified STDs.

With regard to the barring step 728, some methods bar a person from the specified region of the recreational facility for at least one of the following reasons: that person has not been medically screened 710, that person has not given 724 permission to share results of their medical screening with people in the collection. Some methods bar 728 prostitutes and/or other sex workers, regardless of whether they have been medically screened. In addition to barring entry to regions, some methods include refusing 738 to disclose medical screening results to a person for at least one of the following reasons: that person has not been medically screened 710, that person has not given 724 permission to share results of their medical screening with people in the collection 702.

For instance, someone who has been screened 710 might still be barred 728 because they refused to cooperate during step 724. That is, they refused to share their medical test results with other people—including in some cases people they've never met or corresponded with—who did give permission 724 to share their screening results. Sharing 730 involves more than merely sharing medical test results with family or close friends, as might be done in a hospital or clinic (as opposed to a recreational facility). Permission to share 730 means giving 824 permission to share medical status with unrelated people who are not part of the facility's medical personnel. A person who has been screened 710 but refuses to share 730 their screening results will not be part of the collection 702, and might not be allowed 726 to enter region 114 and interact with other patrons there. If the region is secured to limit entry to medically screened persons who agreed to share their screening results, then people who do not agree to share their results should not be allowed to enter the secured region, and should not be made privy 730 to the confidential medical status information 512 being shared among those who did agree to share their information 512.

Some methods donate 740 a portion of facility 100 proceeds to programs for sex education, to a battered women's shelter, to sex abuse treatment programs, and/or other charitable efforts. This ability to generate and direct charitable funds can be a socially beneficial result of the invention, in addition to other potential benefits to society such as helping reduce unwanted conceptions, and helping reduce the spread of diseases.

As noted, some facilities 100 are not directed toward sexual activity, but do facilitate 742 other recreational activities. In other facilities the method can include facilitating 744 consensual sexual activity between adults in the recreational region 114. As noted earlier, this can be done in manner that benefits both individuals and society.

The method 700 can be performed by a facility entity, e.g., by employees of a legal entity that operates a facility 100. It describes methods of the invention from the perspective of those who assist patrons. As illustrated in FIG. 8, the invention may also be viewed from the perspective of a patron. Steps which are performed upon a patron (or a potential patron) in methods having steps shown in FIG. 7 can also be viewed from the perspective of the person upon whom they are performed.

For instance, a step 710 of medically screening a person corresponds to a step 810 of being medically screened. Steps 712, 714, 716 of performing particular kinds of medical screening likewise have corresponding steps which are not shown in FIG. 8 but could be shown and numbered 812, 814, 816, in which a person has those particular kinds of medical screening performed on oneself. Step 718 of informing a person of the results of one or more medical screenings has a corresponding step 818 in which one is informed of the results of one or more medical screenings that were performed on one. The step of obtaining permission 724 has a corresponding step of giving such permission 824. The step of being allowed to enter 726 a region has a corresponding step of entering 826 the region. The step of sharing medical screening results with others 730 has a corresponding step of witnessing one's screening results being shared 830, either by oneself directly by one's presence in a secured region, or by speech, or through some other medical screening results sharing means such as a badge 500 or display 238. The steps of facilitating various kinds of recreational activities 742, 744 each have a corresponding step of participating in such recreational activities 842, 844, respectively.

One method of the invention performed by a facility patron comprises being medically screened 810 for at least one STD, entering 826 a secured recreational region in the facility 100, and participating 744 in that region in sexual activities with another person who has also been medically screened 810 for at least one STD, the method further limited and characterized in that none of the step 744 participants have left the secured recreational region between the time their respective screening step 810 was completed and the time they commenced recreational step 744. In an alternate method, the further limit and characterization of the method is instead that none of the step 744 participants have engaged in sexual activity with anyone between the time their respective screening step 810 was completed and the time they commenced recreational step 744.

In the methods illustrated by FIGS. 7 and 8, and in other methods according to the invention, the steps discussed and/or illustrated may be performed in various orders, or concurrently, unless the outcome of one step is needed to commence another step. For example, permission to share medical screening results could be obtained 724 prior to medically screening 710 the person whose permission is obtained, or after, or during the screening. On the other hand, the results of screening 710 must be available before the person screened can be informed 718 of those results, so step 718 must follow step 710, at least with respect to a particular screening. However, step 718 need not immediately follow step 710, because other steps, such as step 722 and/or step 724, could be performed between step 710 and step 718. Likewise, steps 726 and 730 could be performed in the opposite order than shown in FIG. 7, but step 730 requires results from step 710, so step 730 must follow step 710, at least with respect to a particular screening. Corresponding comments apply to FIG. 8.

Steps may also be repeated. For example, a person might be screened 810, 814 for fertility status, informed 818 of that result, then screened 810, 812 for HIV by a medical test, informed 818 of that result, and then screened by physical examination and/or additional tests 810, 812 for several other STDs, informed 818 of those results, after which they give permission 824 to share the results, enter 826 the secured region 114, see 830 their results on the display 238, and begin participating in recreational activities 744. In these and the other methods of the invention, steps may also be omitted, renamed, and/or grouped differently, except as required by the claims and for operable embodiments. In particular, not every step illustrated need be performed in a given method according to the invention.

Some methods illustrated by FIGS. 7 and 8 involve a secured region 114 and the possibility, at least, of sharing status merely by presence in that region. For instance, one such method comprises being medically screened 810, giving permission 824 for those results to be shared with others in a secured region 114, entering 826 the secured region 114, and by one's presence in the secured region 114 implicitly sharing 830 the screening results—only patrons having a specified medical status (and possibly clearly identified facility employees) are allowed to enter the secured region 114. Another such method comprises medically screening 710 people, informing 718 them of the screening results, obtaining 724 their permission to share those results with other people who are not their family and not facility medical personnel, and allowing 726 the medically screened person who has agreed to share screening results to enter a secured region 114, where recreational activities are facilitated 742, 744. Badges and/or a display may or may not be used 732, 734 to share 730 the person's screening results with others in the secured region. Because this region 114 is secured, a person's mere presence in the secured region 114 may be used 736 instead of badges or a display to share 730 that person's medical screening results.

Other methods illustrated by FIGS. 7 and 8 involve a recreational region that is not secured to inhibit entry of people who have not been medically screened. Instead, badges and/or a display are used to share 730 the medical screening results of people in the recreational region who have been medically screened, and those people who do not have a valid badge 500 and do not appear on a medical screening results display 238 are understood by others in the recreational region to be unscreened. The risks of interacting with unscreened people can then be considered by patrons. For instance, one such method comprises medically screening 710 some people, informing 718 them of their results, obtaining 724 their permission to share 730 those results by placing 732 a badge 500 on them and/or by displaying 734 their results on a display 238, and allowing 726 them to enter a recreational region that includes patrons who have not all been medically screened. Another such method comprises being medically screened 810, learning 818 the results, giving 824 implicit permission to share at least part of the medical screening results by accepting and wearing 832 a badge 500 that displays medical screening results 512, entering 826 a recreational region, and interacting 830, 842, 844 there with other people, not all of whom bear a similar badge 500 displaying their own medical screening results.

In some embodiments, the permissioning steps 724, 824 and the result sharing steps 730, 830 are performed in a single action which makes the person's medical status (as found by screening 710, 810) evident in a manner that is linked to that person and readily visible to other people who are within the same room. This may be done by using 732, 832 badges 500, by using 734, 834 a display 238, or by simply being present 736, 836 in a secured region 114, 118. For instance, a person who wears 832 a badge 500 displaying his or her fertility status 512, knowing that it displays that status, in a place and manner that makes the fertility status evident to other people, has implicitly given permission 824 for that status to be shared with those other people, and by the same act does indeed share 830 that status.

In some embodiments of the present invention, entry control steps 726, 728, 826, 828 operate according to a fertility compatibility admission criterion. For instance, a method may admit 726 to a secured region 114 people who meet a fertility compatibility admission criterion and prevent 728 entrance to the secured region of people who do not meet the fertility compatibility admission criterion. One fertility compatibility admission criterion specifies that only persons of a single selected gender should be admitted to the secured region. Another fertility compatibility admission criterion specifies that only infertile persons (regardless of the gender(s) involved) should be admitted to the secured region. Another fertility compatibility admission criterion specifies that for a specified gender only infertile persons of that gender should be admitted to the secured region, while persons of the opposite gender can be admitted regardless of their fertility status. Regardless of the fertility compatibility admission criterion used, such entry control steps can be performed in conjunction with medical screening steps 710, 810 to determine fertility status.

Note that some people, such as intersexuals, do not necessarily fit into approaches that recognize only strictly male and strictly female as genders. Intersexuals are people who have physical characteristics that make them neither clearly and solely male nor clearly and solely female. Their anatomy is either ambiguous, or is a mixture of both male and female anatomy. With respect to fertility status and the present invention, a given intersexual could be a fertile as a male, fertile as a female, both, or neither. Thus, if the fertility compatibility admission criterion specifies that only infertile males should be admitted to the secured region, while females can be admitted regardless of their fertility status, then an intersexual who is infertile as a male and fertile as a female satisfies the admission criterion.

In some embodiments of the present invention, entry control steps 726, 728, 826, 828 operate according to an STD compatibility admission criterion. For instance, a method may admit 726 to a secured region 114 people who meet an STD compatibility admission criterion and prevent 728 entrance to the secured region of people who do not meet the STD compatibility admission criterion. One STD compatibility admission criterion specifies that only persons having a single specified STD status should be admitted to the secured region; two people have the same STD status when any one of the following is true: each person has no STDs as determined by the screening; each person has the same STD as determined by the screening; each person has the same two or more STDs as determined by the screening. Regardless of the STD compatibility admission criterion used, such entry control steps can be performed in conjunction with medical screening steps 710, 810 to determine STD status. STDs not tested for may or may not be present, and do not necessarily alter a person's admissibility. Some embodiments control entry according to both a fertility compatibility admission criterion and an STD compatibility admission criterion.

Criteria for admission to secured region(s) of the facility 100 can be relative to persons already admitted, or admission criteria can be absolute. With regard to fertility, for instance, a relative criterion would admit people based on the fertility status of whoever has already been admitted, e.g., if only a non-fertile male has been admitted then any female may be admitted next, but if a fertile male has been admitted then to maintain fertility compatibility only non-fertile females may then be admitted. By contrast, an absolute criterion does not vary the admissibility of a person based on the status of those already admitted, e.g., a facility could employ an absolute criterion stating that no fertile persons shall be admitted to the secured region(s). Similarly, STD compatibility admissions criteria may be relative (e.g., if the first person in is HIV+ then only admit other HIV+ persons from that point on, while if the first person is HIV− then only admit other HIV− persons from that point on), or they may be absolute (e.g., only admit HIV+ persons to the secured region).

Different admission criteria may be employed by a single facility 100 at different times, e.g., during specified hours and/or on different days. Different admission criteria may be employed in order to increase or maximize revenue, patron count, and/or patron diversity, or to further social goals or public health policies. However, it is generally desirable for embodiments to help decrease the risk of disease and/or unwanted conceptions at least by increasing patrons' knowledge of the specific risks. Embodiments may heighten patron compatibility by providing an environment that helps control (e.g., by medical screening and/or security) access to other people with whom patrons interact, even if patrons then choose to engage in risky behavior.

Some embodiments provide a security escort, secured corridors, and/or other security measures to allow incompatible patrons within a single facility while separating those patrons into groups such that the patrons within a given group are compatible with the other patrons in that group. For instance, a facility 100 may contain both chlamydia-free and chlamydia-positive people who are, however, separated into two groups, namely, a chlamydia-free group and a chlamydia-positive group. More generally, people may be guided and/or physically separated according to their sexual preferences and/or medical status. For example, people could be directed to a portion of the secured facility that is reserved for sexual activities that do not involve penile or lingual penetration of any body orifice of any participant; restrictions that help avoid such penetration while allowing other sexual activities can substantially reduce or minimize the risk of transmitting STDs.

Some fertility compatible combinations of people for sexual activity are clear without medical testing, e.g., no medical test is needed to indicate that sex between people of the same gender, or sex between a man and an obviously pregnant woman, will not lead to another unplanned or unwanted conception. But other conditions that impact fertility are not clear without screening 710, 810 by medical testing and/or credible documentation of previous medical testing or surgery. One cannot tell simply by looking at a person whether they are infertile due to a hysterectomy, an effective vasectomy, menopause, a chronic deficiency in viable sperm, or a long-term implanted contraceptive (hormonal or otherwise, eg., IUD), for example. With the invention one can learn, via a credible third party such as a facility physician or medical technician, or a badge or display controlled by such medical personnel, the fertility status of a potential sexual partner. In a suitably managed facility, one might also gain that knowledge in a way that does not embarrass anyone involved because the screening and result sharing procedures are applied equally to each patron involved, without condemnation or stigma.

As illustrated by FIG. 11, some methods of the invention promote recreational activities along the lines described above. One promotional method 1100 includes a step 1102 of displaying, for a commercial purpose other than news reporting, information indicating that medical screening is used to determine the status of people with respect to at least one of three conditions, namely, sexually transmitted disease status, other contagious disease status, and fertility status; a step 1104 of displaying, for a commercial purpose other than news reporting, information indicating that people whose status has been thus determined by medical screening may then enter a recreational region and participate in recreational activities there; a step 1106 of displaying, for a commercial purpose other than news reporting, information indicating at least one of the following: that persons who have not been medically screened are inhibited from entering the recreational region; that persons who enter the recreational region do so bearing a badge that displays their medical status; that persons who enter the recreational region will there witness their medical status being shared with other people who are not medical personnel and to whom they are not related; a step 1108 of displaying, for a commercial purpose other than news reporting, contact information whereby a person interested in at least part of the aforesaid information can learn more about it or accept a displayed offer to join a facility club or use a facility 100 about which the aforesaid steps display information; and a step 1110 of failing to prominently display with the aforesaid information a true and understandable statement which: identifies patent owners and/or patent licensees of the present patent and/or corresponding patents in other jurisdictions; and states whether the aforesaid information is displayed with the permission of such an owner or licensee. Other promotional methods vary these steps by order, content, and/or omission, but they all serve to promote a facility 100, facility device 238 or 500, and/or facility method described herein.

Legitimate new reports would not fall within the illustrated method 1100 because such reports are done only for the purpose of reporting news. Non-commercial displays of such information by government officials in the course of their official regulatory duties, or by academics for purely research purposes, for example, would not fall within this method 1100 because they are not for commercial purpose. If the patent covers recreational facilities, for instance, in a first jurisdiction but not in a second jurisdiction, then activities solely within the second jurisdiction do not fall within this method 1100. But advertising or otherwise promoting the specified recreational facilities in the first jurisdiction—by web postings, email, direct mail, or local representatives' presentations, for example—would fall within this method 1100 if all claimed limitations are met, even if the facilities themselves are in the second jurisdiction, because the display is made and/or done in the first jurisdiction.

### CONCLUSION

The invention provides medically screened secured recreational facilities, databases for secured or unsecured recreational facilities, recreational facility badges and displays of medical screening and/or other information, and a variety of recreational facility methods. In its various embodiments, the invention can help advance the public good and benefit individuals by reducing the risk of conceiving unwanted children and/or by reducing the risk of spreading a disease through sexual or other activities. Although the invention involves matters on which reasonable and moral people may hold different views, it is technology that neither forces moral decisions nor denies one the opportunity to make such personal decisions. The invention strives instead to provide information so that people can assess particularized risks and make better-informed decisions in their interactions with one another, to their benefit and the benefit of society.

To the extent that people make interaction decisions with potentially serious—even life-threatening or life-creating—consequences based on information provided in or by embodiments of the invention, those embodiments should be subject to careful quality control by the embodiment operators, and subject as well to appropriate regulations and other legal authority. Suitable status indication quality control means should be used with methods, facilities, and devices of the invention. In particular and without limitation, the power to bring legal actions to deter patent infringement or otherwise enforce intellectual property rights associated with the inventive embodiments (e.g., trademarks, copyrights) may provide substantial assistance to quality control efforts by helping deter and/or shut down inadequate or shoddy operations which also infringe.

Although particular embodiments of the present invention are expressly illustrated and described herein, it will be appreciated that discussion of one type of embodiment also generally extends to other embodiment types. For instance, the description of the methods illustrated in FIGS. 7, 8, and 11 also helps describe the facilities shown in FIGS. 1 through 4, 9, and 10, and the devices shown in FIGS. 5 and 6, which can be operated and/or created according to those methods. The facility descriptions likewise help explain not merely other facility embodiments but also the methods and devices, and the device descriptions help explain the methods and facilities. It does not follow that limitations from one embodiment are necessarily read into another. All of the illustrated facilities are secured, for instance, but not every method of the invention requires a secured facility.

Headings are for convenience only; information on a given topic may be found outside the section whose heading indicates that topic. All claims as filed are part of the specification and thus help describe the invention, and repeated claim language may be inserted outside the claims as needed.

The invention may be embodied in other specific forms without departing from its essential characteristics. The described embodiments are to be considered in all respects only as illustrative and not restrictive.

As used herein, terms such as “a” and “the” and designations such as “recreational region” and “medical screening” are inclusive of one or more of the indicated item or step. In particular, in the claims a reference to an item generally means at least one such item is present and a reference to a step means at least one instance of the step is performed.

The scope of the invention is indicated by the appended claims rather than by the foregoing description. All changes which come within the meaning and range of equivalency of the claims are to be embraced within their scope to the full extent permitted by law.

What is claimed and desired to be secured by patent is:

1. A recreational facility, comprising:
a secured recreational region;
a medical screening region;
within the medical screening region, a medical screening means for screening patrons of the recreational facility to determine their medical status with respect to at least one of two sex-related conditions, namely, sexually transmitted disease status and fertility status;
an authentication region;
within the authentication region, an authentication means for authenticating employees of the recreational facility and other authorized persons and for detecting unauthorized persons through a lack of authentication;
physical security means which separate the secured recreational region from the medical screening region and which define a secured path containing the medical screening region and the secured recreational region, the secured path configured to allow patrons of the recreational facility to enter the secured recreational region after passing through the medical screening region and being medically screened there, and to inhibit patrons from entering the secured recreational region without first passing through the medical screening region; and
a security perimeter which encompasses at least the secured recreational region, the medical screening region, and the authentication region, the security perimeter being defined by physical security barriers that operate to direct patrons such that a patron who leaves the regions encompassed by the security perimeter can return to the secured recreational region only after again passing through the medical screening region and again being screened there, and that operate to inhibit unauthorized persons from entering the secured recreational region.

2. The recreational facility of claim 1, further comprising an exit processing region which has an exit at the security perimeter and has an entrance adjoining the secured recreational region, wherein physical security means in the facility operate to direct patrons such that a patron who enters the exit processing region from the secured recreational region can return to the secured recreational region only after again passing through the medical screening region.

3. The recreational facility of claim 1, wherein the secured recreational region comprises at least one of the following:
a lodging region which includes private rooms for patrons to use while they stay overnight in the secured recreational region;
an eating region which includes furniture for patrons to use while they eat food in the secured recreational region;
a dancing region which includes a dance floor for patrons to use while they dance in the secured recreational region.

4. The recreational facility of claim 1, wherein the secured recreational region comprises a physically demarcated preference zone for patrons to use while they participate in recreational activities in the secured recreational region.

5. The recreational facility of claim 1, wherein the secured recreational region comprises a physically demarcated and secured requirements zone for patrons to use while they participate in recreational activities in the secured recreational region.

6. The recreational facility of claim 1, further comprising a locker room outside the secured recreational region with lockable storage to hold personal belongings of patrons before they enter the secured recreational region.

7. The recreational facility of claim 1, further comprising a display that is readily visible inside the secured recreational region, that is not readily visible outside the security perimeter, that is secured to prevent unauthorized changes to the information it displays, and that is capable of displaying medical status information obtained within the medical screening region.

8. The recreational facility of claim 1, wherein the facility's construction and physical security serve to define a secured path in which a reception region is followed by the medical screening region which is followed by the secured recreational region, such that patrons enter the secured recreational region only after passing through the reception region and then passing through the medical screening region.

9. The recreational facility of claim 1, wherein the secured path includes an exit processing region following the secured recreational region, such that patrons exit the facility only through the exit processing region, and having entered the exit processing region are barred from returning to the secured recreational region except by passing again through the reception region and through the medical screening region.

10. The recreational facility of claim 1, wherein the medical screening region comprises a physical examination room, a medical counseling office, and a lab area for processing medical tests of at least one of two sex-related conditions, namely, sexually transmitted disease status and fertility status.

11. A medical screening method to be performed by personnel of a recreational facility for improving recreational activities in the recreational facility, the method comprising the steps of:
providing a medical screening region and a physically separate recreational region, both being in the recreational facility;
medically screening people in the medical screening region to determine their medical status with respect to at least one of three conditions, namely, sexually transmitted disease status, other contagious disease status, and fertility status;
admitting to the recreational region at least two people whose medical status has been thus determined by the medical screening and who have stayed in the recreational facility continually since being screened, such that the recreational region contains at least two medically screened people whose screening results are likely still accurate at the time of admission to the recreational region; and
facilitating recreational activities by the medically screened people within the recreational region.

12. The method of claim 11, wherein the method further comprises barring people from the recreational region by at least one of the following actions:
barring prostitutes from the recreational region regardless of whether they have been medically screened;
barring people from entering the recreational region after traveling through a security perimeter around at least the medical screening region and the recreational region, because they have not been medically screened after their most recent passage through the security perimeter and before their subsequent attempt to enter the recreational region; and/or
barring people from the recreational region because they refuse to share results of their medical screening with people in the recreational region who are not related to them and are not medical personnel.

13. The method of claim 11, wherein the method is performed in a recreational facility having patrons who are not employees of the facility, the medical screening step medically screens the patrons with respect to at least their sexually transmitted disease (STD) status, and the method further comprises allowing medically screened patrons to enter the recreational region only if their STD status will be readily visible to other patrons inside the recreational region.

14. The method of claim 13, wherein the method comprises placing, on a medically screened patron, a badge having an indication of the patron's STD status, thereby making the patron's STD status readily visible to other patrons inside the recreational region.

15. The method of claim 13, wherein the method comprises placing, on a display that is readily visible to patrons inside the recreational region, an indication of the STD status of another patron who has been medically scanned.

16. The method of claim 11, wherein the method is performed in a recreational facility having patrons who are not employees of the facility, the medical screening step medically screens the patrons with respect to at least their sexually transmitted disease (STD) status, and the method further comprises allowing medically screened patrons to enter the recreational region only if they satisfy an STD compatibility criterion which requires that all patrons in the recreational region have a single specified STD status, and two patrons have the same STD status when exactly one of the following is true: each patron has no STDs as determined by the medical screening; each patron has the same STD as determined by the medical screening; each patron has the same multiple STDs as determined by the medical screening.

17. The method of claim 16, wherein the facility has multiple recreational regions subject to different STD compatibility criteria, and the method comprises allowing medically screened patrons to enter a given recreational region only if they satisfy an STD compatibility criterion for that recreational region.

18. The method of claim 11, wherein the method allows into the recreational region both medically screened patrons whose STD medical status is visible on a badge and/or a display, and patrons who have not been medically screened for an STD since their most recent entry into the recreational facility.

19. The method of claim 11, wherein the method is performed in a recreational facility having patrons who are not employees of the facility, the medical screening step medically screens at least some patrons with respect to at least their fertility status, and the method further comprises making the fertility status of at least some medically screened patrons readily visible to other patrons inside the recreational region.

20. The method of claim 19, wherein the method comprises placing, on a medically screened patron, a badge having an indication of the patron's fertility status, thereby making the patron's fertility status readily visible to other patrons inside the recreational region.

21. The method of claim 19, wherein the method comprises placing, on a display that is readily visible to patrons inside the recreational region, an indication of the fertility status of another patron who has been medically scanned.

22. The method of claim 19, wherein the method allows into the recreational region both medically screened patrons whose fertility status is visible on a badge and/or a display, and patrons who have not been medically screened for fertility since their most recent entry into the recreational facility.

23. The method of claim 11, wherein the method is performed in a recreational facility having patrons who are not employees of the facility, the medical screening step medically screens the patrons with respect to at least their fertility status, and the method further comprises allowing medically screened patrons to enter the recreational region only if they satisfy a fertility compatibility criterion which requires that all patrons in the recreational region as a group meet at least one of the following criteria: each patron is the same gender as the other patrons in the recreational region; each patron in the recreational region is infertile; no patron in the recreational region is fertile as a male; no patron in the recreational region is fertile as a female.

24. The method of claim 23, wherein the facility has multiple recreational regions subject to different fertility compatibility criteria, and the method comprises allowing medically screened patrons to enter a given recreational region only if they satisfy a fertility compatibility criterion for that recreational region.

25. The method of claim 11, wherein the method medically screens people both for at least two STDs and to determine their fertility status.

26. A method for reducing risks to be performed by a person seeking use of a recreational facility, the method comprising:
being medically screened to determine one's medical status with respect to at least three sexually transmitted diseases;
giving permission to a facility representative to share medical status results of one's medical screening with other people in a specified region of the recreational facility, the other people being neither medical personnel nor one's family;
entering the specified region of the recreational facility; and
witnessing as one's medical status results are shared with another person who learns those results in the specified region of the recreational facility.

27. The method of claim 26, wherein the method medically screens one to determine one's status with respect to HIV as one of the sexually transmitted diseases.

28. The method of claim 26, wherein the method medically screens one to determine one's status with respect to at least four sexually transmitted diseases.

29. The method of claim 26, wherein the method also medically screens one to determine one's fertility status.

30. The method of claim 29, further comprising the step of participating in recreational activities within the specified region of the recreational facility.

31. The method of claim 29, further comprising the step of viewing from outside the specified region of the recreational facility demographic information about people who are inside the specified region.

32. A recreational facility badge which comprises:
a substrate of durable wearable material;
at least one strap extending from the substrate;
a medical screening result displayed on the substrate visibly indicating results obtained by medically screening a person with respect to at least one of sexually transmitted disease status and fertility status; and
a fastening means for fastening the strap to the badge after wrapping the strap about a portion of the person's body, thereby attaching the badge to that person.

33. The recreational facility badge of claim 32, further comprising at least one of the following anti-counterfeiting components:
a holographic logo displayed on the substrate;
an anti-theft tag fastened to the substrate;
a transponder fastened to the substrate;
an encoded checksum or signature printed on the substrate.

34. The recreational facility badge of claim 32, further comprising a timestamp result displayed on the substrate visibly indicating when the medical screening results were obtained.

35. The recreational facility badge of claim 32, further comprising a bearer limited identification displayed on the substrate visibly indicating physical identifying characteristics of the person to whom the badge is attached.

36. The recreational facility badge of claim 32, further comprising preference assertions displayed on the substrate visibly indicating preferences of the person to whom the badge is attached.

37. The recreational facility badge of claim 32, wherein the badge comprises a tamper-evident component.

38. A recreational facility badge produced and used by a process comprising the steps of:
medically screening an adult person to determine their sexually transmitted disease status;
linking to that person a display of information which indicates their sexually transmitted disease status; and
admitting that person to a recreational region, the recreational region containing other adults who have also been medically screened and who display to one another their sexually transmitted disease status.

39. A badge produced and used by a process according to claim 38, wherein the linking step comprises printing the person's sexually transmitted disease status on a substrate and physically attaching that substrate to the person.

40. A badge produced and used by a process according to claim 38, wherein the linking step comprises printing the person's sexually transmitted disease status on the person's skin.

41. A badge produced and used by a process according to claim 38, wherein the process further comprises medically screening the person to determine their fertility status and linking to that person a display of information which indicates their fertility status, and wherein the admitting step admits that person to a recreational region containing other adults who have also been medically screened and who display to one another both their sexually transmitted disease status and their fertility status.

42. A method of promoting recreational activities, comprising the steps of:
displaying, for a commercial purpose other than news reporting, information indicating that medical screening is used to determine the status of people with respect to at least one of three conditions, namely, sexually transmitted disease status, other contagious disease status, and fertility status;
displaying, for a commercial purpose other than news reporting, information indicating that people whose status has been thus determined by medical screening may then enter a recreational region and participate in recreational activities there;
displaying, for a commercial purpose other than news reporting, information indicating at least one of the following:
that persons who have not been medically screened are inhibited from entering the recreational region;
that persons who enter the recreational region do so bearing a badge that displays their medical status;
that persons who enter the recreational region will there witness their medical status being shared with other people who are not medical personnel and to whom they are not related;
displaying, for a commercial purpose other than news reporting, contact information whereby a person interested in at least part of the aforesaid information can learn more about it; and
failing to prominently display with the aforesaid information a true and understandable statement which:
identifies patent owners and/or patent licensees of the present patent and/or corresponding patents in other jurisdictions; and
states whether the aforesaid information is displayed with the permission of such an owner or licensee.
